(19) United States
(12) Patent Application Publication
(10) Pub. No.: US 2024/0122893 A1
(43) Pub. Date: Apr. 18, 2024

(54) METHODS FOR INHIBITING CORONAVIRUSES USING SULFORAPHANE

(71) Applicant: The Johns Hopkins University, Baltimore, MD (US)

(72) Inventors: Lorraine V. Brando, Baltimore, MD (US); Alvaro A. Ordonez, Baltimore, MD (US); Robert H. Yolken, Baltimore, MD (US); Sanjay K. Jain, Baltimore, MD (US)

(21) Appl. No.: 18/274,560

(22) PCT Filed: Feb. 23, 2022
(86) PCT No.: PCT/US2022/014267

§ 371 Date: Jul. 27, 2023

Related U.S. Application Data
(60) Provisional application No. 63/142,598, filed on Jan. 28, 2021.

(51) Int. Cl. A61K 31/26; A61K 31/706; A61K 45/06; A61P 31/14

(52) U.S. Cl. CPC A61K 31/26; A61K 31/706; A61K 45/06; A61P 31/14

(57) ABSTRACT

Disclosed herein, are methods for treating, preventing or inhibiting a coronavirus infection or a disease or condition associated with a coronavirus infection. The methods can comprise administering to the subject one or more therapeutically effective doses of sulforaphane.

### CROSS REFERENCE TO RELATED APPLICATIONS

This application claims the benefit of U.S. Provisional Application No. 63/142,598, filed on Jan. 28, 2021. The content of this earlier filed application is hereby incorporated by reference herein in its entirety.

### INCORPORATION OF THE SEQUENCE LISTING

The present application contains a sequence listing that is submitted via EFS-Web concurrent with the filing of this application, containing the file name “36406_0023P1_SL.txt” which is 4,096 bytes in size, created on Jan. 20, 2022, and is herein incorporated by reference in its entirety.

### STATEMENT REGARDING FEDERALLY FUNDED RESEARCH

This invention was made with government support under grant numbers AI149760, AI145435, EB020539, and AI153349 awarded by the National Institutes of Health. The government has certain rights in the invention.

### FIELD OF THE INVENTION

The present invention relates to compositions and methods for treating, preventing and inhibiting a coronavirus infection.

### BACKGROUND

COVID-19, currently considered a worldwide pandemic, is caused by the highly contagious coronavirus SARS-CoV-2. Limited medications have been approved for emergency use but they are inadequate in terms of efficacy and terms of use. Thus, alternative therapies are needed.

### SUMMARY

Disclosed herein are methods of treating a subject having a coronavirus infection, the methods comprising administering to the subject one or more therapeutically effective doses of sulforaphane.

Disclosed herein are methods of treating or preventing COVID-19, in a subject, the methods comprising administering to the subject one or more therapeutically effective doses of sulforaphane.

Disclosed herein are methods of preventing or inhibiting a coronavirus infection in a subject, the methods comprising administering to the subject one or more therapeutically effective doses of sulforaphane.

Disclosed herein are methods of inhibiting replication of a coronavirus in a subject having a coronavirus infection, the methods comprising administering to the subject one or more therapeutically effective doses of sulforaphane.

Disclosed herein are methods of inhibiting replication of a coronavirus in a cell, the methods comprising contacting the cell infected with a coronavirus with one or more therapeutically effective doses of sulforaphane.

Disclosed herein are methods of inhibiting, treating or preventing a coronavirus infection in a subject, the methods comprising administering to the subject having said infection a plurality of therapeutically effective doses of sulforaphane.

Disclosed herein are kits for use in treating a subject suffering from a coronavirus infection, said kits comprising: one or more therapeutically effective doses of (a) sulforaphane; and (b) remdesivir

Disclosed herein are kits for use in preventing or inhibiting a coronavirus infection in a subject, said kits comprising: one or more therapeutically effective doses of (a) sulforaphane; and (b) remdesivir.

Disclosed herein are kits for use in inhibiting replication of a coronavirus infection in a subject, said kits comprising: one or more therapeutically effective doses of (a) sulforaphane; and (b) remdesivir.

### BRIEF DESCRIPTION OF THE DRAWINGS

FIGS. 1A-F show antiviral effects of SFN against HCoV-OC43. Median effect plots and dose-effect curves calculated for (FIG. 1A) Vero C1008 cells infected with HCoV-OC43 after a 1-2 h incubation with increasing concentrations of SFN. FIG. 1B shows MRC-5 cells infected with HCoV-OC43 after a 1-2h incubation with increasing concentrations of SFN. FIG. 1C shows Vero C1008 cells infected with HCoV-OC43 over 24 h, after which they were incubated with SFN. FIG. 1D shows Vero C1008 cells incubated with SFN for 24 h, after which the drug was removed, and the cells were infected with HCoV-OC43. FIG. 1E shows Vero C1008 cells infected with HCoV-OC43 after a 1-2 h incubation with increasing concentrations of remdesivir. FIG. 1F shows a normalized isobologram showing combination index (CI) for combinations of various doses. Antiviral data displayed in red (and circles); anti-host cell activity (cytotoxicity) displayed in blue (and squares). Synergism (CI<1); Additive effect (CI=1); Antagonism (CI>1); SFN, Sulforaphane; RDV, remdesivir. Dotted lines represent 95% confidence interval. Experiments performed a minimum of 2 times (range=2-7), 3-6 replicates within each experiment, experiment shown in FIG. 1D was performed once.

FIGS. 2A-F show antiviral effects of SFN against SARS-CoV-2. Median effect plot and dose-effect curves calculated for (FIG. 2A) Vero C1008 cells infected with SARS-CoV-2/Wuhan-Hu-1 after 1-2 h incubation with increasing concentrations of SFN. FIG. 2B shows Vero C1008 cells infected with SARS-CoV-2/Wuhan-Hu-1 for 24 h and then incubated with SFN. Antiviral data displayed in red (and circles); anti-host cell activity (cytotoxicity) displayed in blue (squares). FIG. 2C shows the antiviral activity in human Caco-2 cells that was determined by measuring viral RNA by qPCR. The cells were incubated with SFN for 1 h before viral inoculation. FIG. 2D shows the effects of SFN evaluated in Vero C1008 cells exposed to drug for 1 h followed by viral inoculation. A reference strain (USA-WI1/2020) and two 614G+ clinical strains of SARS-CoV-2 were evaluated for CPE using a bioluminescence readout FIG. 2E shows the effects of SFN and remdesivir evaluated in Vero C1008 cells exposed to drug for 1 h followed by viral inoculation. FIG. 2F shows a normalized isobologram showing combination index (CI) for combinations of various doses of SFN and remdesivir. Synergism (CI<1); Additive effect (CI=1); Antagonism (CI>1); SFN, Sulforaphane; RDV, Remdesivir. Dotted lines represent 95% confidence interval. Experiments performed a minimum of 2 times (range=2-7), 3-6 replicates within each experiment, experiment shown in FIG. 2E was performed once.

FIGS. 3A-H show SFN treatment in SARS-CoV-2 infected K18-hACE2 mice. FIG. 3A shows xix- to eight-week-old male K18-hACE-transgenic mice were randomly distributed among treatment groups and inoculated intranasally with SARS-CoV-2/USA/WI1/2020 or vehicle. FIG. 3B shows that four days post inoculation, there was a marked weight loss in the infected groups, although there was significantly less weight loss in the SFN treated animals. By day 6 post inoculation, the SFN-treated animals had lost 7.5% less bodyweight compared to infected untreated controls (one-way ANOVA, ***P<0.0001). Data from three independent experiments, uninfected (n=8), infected untreated (n=16), infected treated (n=14). FIG. 3C shows bronchoalveolar lavage (BAL) total protein quantification, determined as a surrogate for lung injury, measured 6 days post-infection. Infected untreated animals had significantly higher total protein compared to the infected treated group (one-way ANOVA, ***P<0.0001). Data from three independent experiments, uninfected (n=8), infected untreated (n=16), infected treated (n=14). FIG. 3D show the viral load in the BAL, as determined by qPCR, was significantly higher in infected untreated animals compared to the infected treated group (Mann-Whitney U test, two-tailed. *P=0.036). Data from two independent experiments, infected untreated (n=8), infected treated (n=9). FIG. 3E shows the viral load in the lungs of infected treated animals, represented as the SARS-CoV-2 N protein copies normalized to Pol2Ra, had a 1.5 log reduction compared to infected untreated controls (Mann-Whitney U test, two-tailed, **P=0.004). Data from two independent experiments, infected untreated (n=11), infected treated (n=9). FIG. 3F shows hematoxylin and eosin (H&E) staining and immunostaining for SARS-CoV-2 spike protein, of histological sections of the lungs of representative uninfected control, infected untreated, and infected treated mice. Regions of the lung anatomy where alveolar and peribronchiolar inflammation was assessed are highlighted in boxes. Images show low (left panels; scale bar, 1 mm) and high-power magnification (right panels; scale bar, 50 μm) of the same tissue section. FIG. 3G shows histopathological severity scoring that was evaluated according to the pathological changes as described herein in Example 2. Data from one independent experiment, infected untreated (n=8), infected treated (n=5). Mann-Whitney U test, two-tailed, **P=0.0008. FIG. 3H shows the quantification of the SARS-CoV-2 spike protein immunostaining showed a 4.41× lower % area in the lungs of SFN-treated mice compared to infected untreated controls (P=0.01). Data from one independent experiment, uninfected (n=4), infected untreated (n=8), infected treated (n=5). One-way ANOVA, *P<0.05, **P<0.005. the data in this figure are represented as mean standard deviation. Each dot represents one animal.

FIGS. 4A-D show the effects of SFN treatment in the immune response. FIG. 4A shows Uniform Manifold Approximation and Projection (UMAP) that was used to visualize the immune cell populations within the spleen and lung of uninfected (grey), infected untreated (blue), and infected treated (red) mice. The corresponding immune cell populations are presented in multiple colors in the panels on the right. FIG. 4B is a summary of immune cell frequencies out of total CD45+ immune cells in spleen and lung of infected treated or untreated mice. FIG. 4C shows the total cell count of indicated immune cell subset per spleen. Each dot represents one mouse. FIG. 4D shows the total cell count of indicated immune cell subset per lung. Each dot represents one mouse, data from one independent experiment Bars represent mean values. DCs, dendritic cells; NK, natural killer, M-MDSC, mononuclear myeloid-derived suppressor cells. Statistical comparisons made with two-way ANOVA, *P<0.05, **P<0.01.

FIGS. 5A-E shows the functional characterization of the immune response after SFN treatment FIG. 5A shows myeloid cell subsets that are shown as percent of total CD45+ immune cells within the lung. FIG. 5B shows alveolar macrophages (AM) after stimulation with protein transport inhibitors. MFI, mean fluorescent intensity. FIG. 5C shows cytokine expression in alveolar macrophages after stimulation with protein transport inhibitors. FIG. 5D T cells were stained immediately ex vivo without further stimulation and evaluated for the expression of Ki67, PD1 and MHC-II. Percent of CD8+ or CD4+ T cells from spleen or lung expressing indicated marker are shown. FIG. 5E shows the percent of CD8+ or CD4+ T cells from spleen or lung expressing indicated marker after stimulation with PMA/ionomycin. Each dot represents one mouse, data from one independent experiment. Data represented as mean standard error of mean. n=4 uninfected, n=5 infected SFN-treated, and n=8 infected untreated animals. Statistical comparisons made with one-way ANOVA, *P<0.05, **P<0.01, ***P<0.001, ****P<0.0001.

FIGS. 6A-B show antiviral activity of SFN on NRF2 knockdown (KD) cells. NRF2 was silenced from Caco-2 cells by CRISPR/Cas9 to reduce the expression of NRF2. FIG. 6A shows Westem blot determination of NRF2, in total cell lysate of Caco-2 cells, control, and NRF2 KD, treated as described herein. O-actin was used as loading control. FIG. 6B shows control and NRF2 KD cells that were treated with SFN (5 μM) or DMSO (vehicle control) over 1-2 h. Subsequently, the cells were infected with SARS-CoV-2/USA-WI1/2020 and incubated over 48 h. Culture supematants were collected and processed for quantification of SARS-CoV-2 copies by qRT-PCR. A significant reduction in viral load was observed in cells treated with SFN in both control and NRF2 KD cells (P<0.001). There was no significant difference in the viral load with or without SFN treatment in NRF2 KD cells compared to control cells (P≥0.54). Data representative of three independent experiments. Statistical comparisons with one-way ANOVA with Tukey's multiple comparisons test.

FIG. 7 shows hACE2 expression in lung tissues. The expression of hACE2 in the lung of K18-hACE2 mice infected with SARS-CoV-2 and treated with SFN was primarily in the airway epithelia. The lung section of representative mouse is shown.

FIG. 8 shows pulmonary viral burden. The viral load in the lungs of infected treated animals, represented as the total SARS-CoV-2 N protein copies, had a 1.46 log reduction compared to infected untreated controls (Mann-Whitney U test, two-tailed, P=0.1308). Data not normalized to Pol2Ra. Data from two independent experiments, infected untreated (n=11), infected treated (n-9).

FIGS. 9A-C show histology and SARS-CoV-2 spike protein immunostaining. Low magnification (scale bar, 1 mm) and the corresponding high magnification areas (scale bar, 50 μm) of Hematoxylin and eosin staining (top panels) and SARS-CoV-2 spike protein immunostaining (bottom panels) of the lungs of (FIG. 9A) uninfected, (FIG. 9B) infected SFN-treated, and (FIG. 9C) infected untreated animals.

FIGS. 10A-B show functional markers of immune response in the lung. Flow cytometric analysis of pulmonary alveolar macrophages (FIG. 10A) and interstitial macrophages (FIG. 10B). MFI, mean fluorescent intensity. Data represented as mean standard error of mean. n=4 uninfected, n=5 infected SFN-treated, and n=8 infected untreated animals. Statistical comparisons made with one-way ANOVA, *P<0.05, **P<0.01, ***P<0.001.

FIGS. 11A-B show functional markers of macrophages in the bronchoalveolar lavage. Flow cytometric analysis of alveolar macrophages (FIG. 11A) and interstitial macrophages (FIG. 11b) of the bronchoalveolar lavage. MFI, mean fluorescent intensity. Data represented as mean standard error of mean. n=4 uninfected, n=5 infected SFN-treated, and n=8 infected untreated animals. Statistical comparisons made with one-way ANOVA, *P<0.05, **P<0.01, ***P<0.001.

FIGS. 12A-C show functional characterization of T cells. Flow cytometric analysis of T cells after stimulation with PMAlionomycin in the spleen (FIG. 12A), lung (FIG. 12B), and bronchoalveolar lavage (FIG. 12C). MFI, mean fluorescent intensity. Data represented as mean standard error of mean. n=4 uninfected, n=5 infected SFN-treated, and n=8 infected untreated animals. Statistical comparisons made with one-way ANOVA, *P<0.05, **P<0.01, ***P<0.001, ****p<0.0001.

### DETAILED DESCRIPTION

The present disclosure can be understood more readily by reference to the following detailed description of the invention, the figures and the examples included herein.

Before the present methods and compositions are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

As used in the specification and the appended claims, the singular forms “a,” “an” and “the” include plural referents unless the context clearly dictates otherwise.

The word “or” as used herein means any one member of a particular list and also includes any combination of members of that list.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “subject” can refer to the target of administration, e.g., a human. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In some aspects, a subject is a mammal. In some aspects, a subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been identified with having a suspected coronavirus exposure, having a coronavirus infection or being susceptible to a coronavirus infection, such as, for example, prior to the administration step.

As used herein, the term “comprising” can include the aspects “consisting of” and “consisting essentially of.”

A “SARS virus protein” refers to any protein of any SARS virus strain or its functional equivalent as defined herein. Thus, the invention includes, but is not limited to, SARS polymerase, the S (spike) protein, the N (nucleocapsid) protein, the M (membrane) protein, the small envelope E protein, the Nsp (non-structural proteins), and their functional equivalents.

As used herein, the term “contacting” can refer to the placement in direct physical association; includes both in solid and liquid form. “Contacting” is often used interchangeably with “exposed.” In some aspects, “contacting” refers to delivering or exposing a cell to a molecule (such as sulforaphane).

As used herein, the terms “synergy”, “synergism” or “synergistic” mean more than the expected additive effect of a combination. A synergistic effect may be attained when the active ingredients are: (1) co-formulated and administered or delivered simultaneously in a combined, unit dosage formulation; (2) delivered by alternation or in parallel as separate formulations; or (3) by some other regimen.

As used herein, the term “treating” refers to partially or completely alleviating, ameliorating, relieving, delaying onset of, inhibiting or slowing progression of, reducing severity of, and/or reducing incidence of one or more symptoms or features of a particular disease, disorder, and/or condition. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. For example, the disease, disorder, and/or condition can be a coronavirus infection or the disease, disorder, and/or condition can be associated with a coronavirus infection (e.g., COVID-19).

As used herein, the terms “inhibit,” “inhibiting,” and “inhibition” mean to diminish or decrease an activity, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in an aspect, the inhibition or reduction can be a 10, 20, 30, 40, 50, 60, 70, 80, 90, 100 percent, or any amount of reduction in between as compared to native or control levels. In an aspect, the inhibition or reduction is 10-20, 20-30, 30-40, 40-50, 50-60, 60-70, 70-80, 80-90, or 90-100 percent as compared to native or control levels. In an aspect, the inhibition or reduction is 0-25, 25-50, 50-75, or 75-100 percent as compared to native or control levels. Further, the terms, “inhibit” or “inhibiting” mean decreasing viral colonization from the amount of colonization that would occur without treatment and/or causing an infection to decrease. Inhibiting also include causing a complete regression of the colonization.

As used herein, “modulate” is meant to mean to alter, by increasing or decreasing.

As used herein, “prevent” is meant to mean minimize the chance that a subject who has an increased susceptibility for developing an infection will develop an infection. In some aspects, the subject has an increased susceptibility for developing an infection because the subject was exposed to one or more viruses disclosed herein. In some aspects, the term “prevent” can also mean minimizing the chance that a subject who has been exposed to one or more viruses disclosed herein will develop a disease, disorder, and/or condition associated with a coronavirus infection (e.g., COVID-19).

As used herein, “treat” is meant to mean administer a compound, composition or molecule of the invention to a subject, such as a human or other mammal (for example, an animal model), that has an infection, in order to prevent or delay a worsening of the effects of the disease or condition, or to partially or fully reverse the effects of the disease.

As used herein, “effective amount” of a compound is meant to mean a sufficient amount of the compound to provide the desired effect. The exact amount required will vary from subject to subject, depending on the species, age, and general condition of the subject, the severity of disease (or underlying genetic defect) that is being treated, the particular compound used, its mode of administration, and the like. Thus, it is not possible to specify an exact “effective amount.” However, an appropriate “effective amount” may be determined by one of ordinary skill in the art using only routine experimentation.

Severe Acute Respiratory Syndrome Coronavirus 2 (SARS-CoV-2), the cause of coronavirus disease 2019 (COVID-19), has incited a global health crisis and has resulted in substantial global morbidity and mortality. Currently, there are limited therapeutic options for the prevention and treatment of SARS-CoV-2 infections. No orally bioavailable medications are available to prevent infections with coronaviruses including those that cause COVID-19 or even seasonal colds. While an unprecedented effort has led to the development of highly effective vaccines, many people remain vulnerable to developing severe disease due to inadequate accessibility or unwillingness to be vaccinated, as well as poor immune responses in certain populations. Other therapeutic approaches have also been developed for COVID-19, including early treatments with monoclonal antibodies against SARS-CoV-2 (Taylor, P. C., et al. Nat Rev Immunol 21, 382-393 (2021)), convalescent plasma (Sullivan, D. J., et al. medRxiv, 2021.2012.2010.21267485 (2021)); and Gordon, O., et al. JCI Insight (2021). December 2; e151518) and antivirals (Beigel, J. H., et al. N Engl J Med 383, 1813-1826 (2020)). Immunomodulators have also been utilized to modify disease and prevent mortality (Group, T.W.RE.A.f.C.-T.W. JAMA 326, 499-518 (2021)). Early intervention after symptom onset has been shown to be most effective in preventing severe disease and hospitalizations (Gupta, A., et al. NEng1 J Med 385, 1941-1950 (2021); and Jayk Bemal, A., et al. N Engl J Med (2021). December 16; NEJMoa2116044). It is imperative that the scientific community quickly find more medications to build a therapeutics armory for healthcare providers to safely try for the prevention and/or treatment of COVID-19. Therefore, a therapy that is readily available and easily administered to patients needs to be developed. Among the direct-acting antivirals, molnupiravir and ritonavir-boosted nirmatrelvir (paxlovid) are oral agents currently authorized by the United States Food and Drug Administration for the treatment of patients with COVID-19 (Jayk Bernal, A., et al. N Engl J Med (2021). December 16; NEJMoa2116044; Owen. D. R., et al. Science 374, 1586-1593 (2021); and Cox, R M., et al. Nat 544 Microbiol 6, 11-18 (2021)). Additional oral antiviral therapeutics are urgently needed to prevent more severe disease, hospitalization, and death.

Sulforaphane, a major principle of broccoli sprout extract (and other cruciferous vegetables), is widely available as a dietary supplement for humans. It is an isothiocyanate derived from enzymatic hydrolysis of its precursor glucoraphanin. The antiviral activity of sulforaphane (SFN), the principal biologically active phytochemical derived from glucoraphanin, the precursor present in high concentrations in in broccoli (Brassica oleracea italica) and other cruciferous vegetables was evaluated. As described herein SFN inhibited in vitro replication of four strains of SARS-CoV-2 as well as that of the seasonal coronavirus HCoV-OC43. Further, SFN and remdesivir interacted synergistically to inhibit coronavirus infection in vitro. Prophylactic administration of SFN to K18-hACE2 mice prior to intranasal SARS-CoV-2 infection significantly decreased the viral load in the lungs and upper respiratory tract and reduced lung injury and pulmonary pathology compared to untreated infected mice. SFN treatment diminished immune cell activation in the lungs, including significantly lower recruitment of myeloid cells and a reduction in T cell activation and cytokine production.

SFN is a potent activator of the transcription factor nuclear factor erythroid 2-related factor 2 (NRF2), with antioxidant and anti-inflammatory effects (Yagishita, Y., et al. Molecules 24 (2019); Sulforaphane glucosinolate. Monograph. Altem Med Rev 15, 352-360 (2010); and Mahn, A. & Castillo, A. Molecules 26, 752 (2021)). Treatment with SFN increased phagocytic activity of alveolar macrophages 13 and reduced lung injury in animal models of acute respiratory distress syndrome (ARDS) 14. SFN also decreased the levels of IL-6 and viral load in human subjects infected with live attenuated influenza virus (Noah, T. L., et al. PLoS One 9, e98671 (2014); and Houghton, C.A. OxidMed Cell Longev 2019, U.S. Pat. No. 2,716,870 (2019)). Numerous clinical trials utilizing SFN have demonstrated favorable pharmacokinetics after oral dosing and document excellent tolerability and safety (Yagishita, Y., et al. Molecules 24 (2019); Cuadrado, A., et al. Trends Pharmacol Sci 41, 598-610 (2020); Axelsson, A S., et al. Sci Transl Med 9, eaah4477 (2017); and Dickerson, F., et al. Schizophr Res 231, 142-144 (2021)).

As disclosed herein, SFN is efficacious against human coronaviruses. As disclosed herein, SFN inhibits in vitro seasonal coronavirus HCoV-OC43 and SARS-CoV-2 infections of mammalian host cells and has a synergistic interaction with remdesivir. In addition, SFN reduces viral load and pulmonary pathology in a mouse model of SARS-CoV-2 infection. The results disclosed herein show that SFN can be used for the prevention or treatment in a subject with COVID-19 as well as other coronavirus infections or diseases.

Described herein are compositions comprising sulforaphane, a major isothiocyanate compound present in cruciferous vegetables, and methods of using said compositions to efficiently inhibit the replication of SARS-CoV-2 and a seasonal coronavirus, OC43.

Coronaviruses are a group of RNA viruses that can cause respiratory tract infections that can range from mild to lethal. Mild illnesses include the common cold. Lethal illnesses to date include SARS, MERS, and COVID-19. Coronaviruses constitute the subfamily Orthocoronavirinae, in the family Coronaviridae, order Nidovirales, and realm Riboviria. They are enveloped viruses with a positive-sense single-stranded RNA genome and a nucleocapsid of helical symmetry that is wrapped in an icosahedral protein shell. The genome size of coronaviruses ranges from approximately 26 to 32 kilobases. Club-shaped spikes that project from their surface are characteristic of coronaviruses.

Coronavirus (CoV) is the common name for Coronaviridae. In humans, CoV causes respiratory infections, which are typically mild but can be lethal in rare forms such as SARS (severe acute respiratory syndrome)-CoV, MERS (Middle East Respiratory Syndrome)-CoV, and COVID-19. CoV has a nucleocapsid of helical symmetry and the genome size ranges from about 26 to about 32 kilobases. Other examples of human CoV (HCoV) include, but are not limited to HCoV-229E, HCoV-NL63, HCoV-OC43, and HCoVHKU1. The envelope of CoV carries three glycoproteins: S—spike protein: receptor binding, cell fusion, major antigen; E—Envelope protein: small, envelope-associated protein; and M—Membrane protein: transmembrane—budding & envelope formation. In a few types of CoV, there is a fourth glycoprotein: HE—hemagglutinin-esterase. The genome has a 5′ methylated cap and 3′ poly-A and functions directly as mRNA. Entry of the CoV into a human cell occurs via endocytosis and membrane fusion; and replication occurs in the cell's cytoplasm. CoV are transmitted by aerosols of respiratory secretions, by the fecal-oral route, and by mechanical transmission. Most virus growth occurs in epithelial cells. Occasionally the liver, kidneys, heart or eyes may be infected, as well as other cell types such as macrophages. In cold-type respiratory infections, growth appears to be localized to the epithelium of the upper respiratory tract. Coronavirus infection is very common and occurs worldwide. The incidence of infection is strongly seasonal, with the greatest incidence in children in winter. Adult infections are less common. The number of coronavirus serotypes and the extent of antigenic variation is unknown. Re-infections appear to occur throughout life, implying multiple serotypes (at least four serotypes of HCoV-OC43 are known) and/or antigenic variation, hence, the prospects for immunization against all serotypes with a single vaccine is highly unlikely. SARS is a type of viral pneumonia, with symptoms including fever, a dry cough, dyspnea (shortness of breath), headache, and hypoxaemia (low blood oxygen concentration). Typical laboratory findings include lymphopaenia (reduced lymphocyte numbers) and mildly elevated aminotransferase levels (indicating liver damage). Death may result from progressive respiratory failure due to alveolar damage. The typical clinical course of SARS involves an improvement in symptoms during the first week of infection, followed by a worsening during the second week. A substantial need remains for effective treatments (compositions and methods) against human CoV.

Severe Acute Respiratory Syndrome Coronavirus 2 (SARS-CoV-2), the cause of coronavirus disease-2019 (COVID-19), has incited a global health crisis. While several vaccine candidates have shown encouraging efficacy for prevention of infection by SARS-CoV-2, the global administration for the general population is still limited. Meanwhile, infection cases are rapidly increasing worldwide. Currently, there are no available medications for prophylaxis for those exposed to SARS-CoV-2, and limited therapeutic options for those who develop COVID-19. Approved anti-COVID-19 treatments such as remdesivir and immunomodulatory therapies cannot be delivered orally, making them impractical for viral transmission control.

As described herein, the antiviral activity of sulforaphane (SFN), a multi-functional, orally available, phytochemical known to be a potent NRF2 activator with very few side effects, was evaluated. Sulforaphane inhibited in vitro virus-associated cell death in cells infected with either SARS-CoV-2 or seasonal coronavirus HCoV-OC43. SFN treatment before SARS-CoV-2 infection of K18-hACE2 mice significantly reduced the viral load in the upper respiratory tract and improved pulmonary pathology compared to untreated controls. These results suggest that SFN can be a therapy for prevention of coronavirus infection or treatment of early disease.

### METHODS OF TREATMENT

Disclosed herein are methods of treating a subject having a coronavirus infection (e.g., SARS-CoV-2). In some aspects, the methods can comprise administering to the subject one or more therapeutically effective doses of sulforaphane. In some aspects, sulforaphane can inhibit replication of the coronavirus. In some aspects, the methods can further comprise administering to the subject one or more therapeutically effective doses of remdesivir. In some aspects, the subject is infected or has previously been infected with the coronavirus.

Disclosed herein are methods of treating or preventing COVID-19 in a subject. Also disclosed herein are methods of treating or preventing a disease resulting from a coronavirus infection. In some aspects, the disease resulting from a coronavirus infection is COVID-19. In some aspects, the methods can comprise administering to the subject one or more therapeutically effective doses of sulforaphane. In some aspects, sulforaphane can inhibit replication of the coronavirus. In some aspects, the methods can further comprise administering to the subject one or more therapeutically effective doses of remdesivir. In some aspects, the subject is not infected and has not previously been infected with the coronavirus. In some aspects, the subject is at risk of being infected with the coronavirus. In some aspects, the subject is infected or has previously been infected with the coronavirus.

Disclosed herein are methods of preventing or inhibiting a coronavirus infection in a subject. In some aspects, the methods can comprise administering to the subject one or more therapeutically effective doses of sulforaphane. In some aspects, sulforaphane can inhibit replication of the coronavirus. In some aspects, the methods can further comprise administering to the subject one or more therapeutically effective doses of remdesivir. In some aspects, the subject is not infected and has not previously been infected with the coronavirus. In some aspects, the subject is at risk of being infected with the coronavirus. In some aspects, the subject is infected or has previously been infected with the coronavirus.

Disclosed herein are methods of preventing or inhibiting a respiratory infection in a subject. In some aspects, the methods can comprise administering to the subject one or more therapeutically effective doses of sulforaphane. In some aspects, sulforaphane can inhibit replication of the respiratory virus. In some aspects, the methods can further comprise administering to the subject one or more therapeutically effective doses of remdesivir. In some aspects, the subject is not infected and has not previously been infected with the respiratory virus. In some aspects, the subject is at risk of being infected with the respiratory virus. In some aspects, the subject is infected or has previously been infected with the respiratory virus.

Disclosed herein are methods of inhibiting replication of a coronavirus in a subject having a coronavirus infection. In some aspects, the methods can comprise administering to the subject one or more therapeutically effective doses of sulforaphane. In some aspects, sulforaphane can inhibit replication of the coronavirus. In some aspects, the methods can further comprise administering to the subject one or more therapeutically effective doses of remdesivir. In some aspects, the subject is not infected and has not previously been infected with the coronavirus. In some aspects, the subject is at risk of being infected with the coronavirus. In some aspects, the subject is infected or has previously been infected with the coronavirus.

Disclosed herein are methods of inhibiting replication of a respiratory virus in a subject having a respiratory infection. In some aspects, the methods can comprise administering to the subject one or more therapeutically effective doses of sulforaphane. In some aspects, sulforaphane can inhibit replication of the respiratory virus. In some aspects, the methods can further comprise administering to the subject one or more therapeutically effective doses of remdesivir. In some aspects, the subject is not infected and has not previously been infected with the respiratory virus. In some aspects, the subject is at risk of being infected with the respiratory virus. In some aspects, the subject is infected or has previously been infected with the respiratory virus.

Disclosed herein are methods of inhibiting replication of a coronavirus in a cell. In some aspects, the methods can comprise contacting the cell infected with the coronavirus with one or more therapeutically effective doses of sulforaphane. In some aspects, the methods can comprise delivering to the cell infected with the coronavirus one or more therapeutically effective doses of sulforaphane. In some aspects, the methods can further comprise contacting the cell or delivering to the cell infected with the coronavirus one or more therapeutically effective doses of remdesivir.

Disclosed herein are methods of inhibiting replication of a respiratory virus in a cell. In some aspects, the methods can comprise contacting the cell infected with the respiratory virus with one or more therapeutically effective doses of sulforaphane. In some aspects, the methods can comprise delivering to the cell infected with the respiratory virus one or more therapeutically effective doses of sulforaphane. In some aspects, the methods can further comprise contacting the cell or delivering to the cell infected with the respiratory virus one or more therapeutically effective doses of remdesivir.

Disclosed herein are methods of inhibiting, treating or preventing a coronavirus infection in a subject. Also disclosed herein are methods of inhibiting, treating or preventing a respiratory infection in a subject. In some aspects, the methods can comprise administering to the subject having said infection a plurality of therapeutically effective doses of sulforaphane. In some aspects, sulforaphane can inhibit replication of the coronavirus. In some aspects, the methods can further comprise administering to the subject one or more therapeutically effective doses of remdesivir. In some aspects, the subject is not infected and has not previously been infected with the coronavirus. In some aspects, the subject is at risk of being infected with the coronavirus. In some aspects, the subject is infected or has previously been infected with the coronavirus. In some aspects, the plurality of therapeutically effective doses of sulforaphane can be one or more doses administered per day for two or more days per week. In some aspects, the dosing can be continued for one or more weeks per month. In some aspects, the dosing can be continued for one or more months per year.

In some aspects, sulforaphane can be administered to the subject immediately after infection or any time within one day to 5 days after infection or at the earliest time after diagnosis of infection with the coronavirus.

In some aspects, sulforaphane can be administered to the subject as a primary antiviral therapy, adjunct antiviral therapy, or a co-antiviral therapy, or wherein the administration comprises separate administration or coadministration of sulforaphane with at least one other antiviral composition or with at least one other composition for treating one or more symptoms associated with said coronavirus infection. In some aspects, the at least one other antiviral composition can be remdesivir.

In some aspects, the subject can be a human subject. In some aspects, the cell can be a mammalian cell. In some aspects, the mammalian cell can be a human cell. In some aspects, the subject is not infected and has not previously been infected with the coronavirus. In some aspects, the subject is at risk of being infected with the coronavirus. In some aspects, the subject has been exposed to or is suspected of being exposed to a coronavirus. In some aspects, the subject has not been exposed to or is not suspected of being exposed to a coronavirus. In some aspects, the subject has been exposed to or is suspected of being exposed to a respiratory virus. In some aspects, the subject has not been exposed to or is not suspected of being exposed to a respiratory virus. In some aspects, the subject has an active infection. In some aspects, the subject has had a prior infection.

Any of the methods described herein can include the step of determining whether or not the subject has a viral infection; indicating administration of sulforaphane; administering an initial dose of sulforaphane to the subject according to a prescribed initial dosing regimen for a period of time; periodically determining the adequacy of subject's clinical response and/or therapeutic response to treatment with sulforaphane; and if the subject's clinical response and/or therapeutic response is adequate, then continuing treatment with sulforaphane as needed until the desired clinical endpoint is achieved; or if the subject's clinical response and/or therapeutic response are inadequate at the initial dose and initial dosing regimen, then escalating or deescalating the dose until the desired clinical response and/or therapeutic response in the subject is achieved.

Treatment of the subject with sulforaphane can be continued as needed. The dose or dosing regimen can be adjusted as needed until the patient reaches the desired clinical endpoint(s) such as a reduction or alleviation of specific symptoms associated with the viral infection. Determination of the adequacy of clinical response and/or therapeutic response can be conducted by a clinician familiar with viral infections.

In some aspects, the coronavirus can be pathogenic to humans. In some aspects, the coronavirus can be severe acute respiratory syndrome coronavirus 2(SARS-CoV-2), severe acute respiratory syndrome (SARS), middle east respiratory syndrome (MERS), human coronavirus 229E, human coronavirus NL63, Miniopterus bat coronavirus 1, Miniopterus bat coronavirus HKU8, porcine epidemic diarrhea virus, Rhinolophus bat coronavirus HKU2, Scotophilus bat coronavirus 512, bovine coronavirus, human coronavirus OC43, human coronavirus HKU1, murine coronavirus, Pipistrellus bat coronavirus HKU5, Rousettus bat coronavirus HKU9, Tylonycteris bat coronavirus HKU4, hedgehog coronavirus 1, infectious bronchitis virus, beluga whale coronavirus SW1, infectious bronchitis virus, Bulbul coronavirus HKU11, pangolin coronavirus, porcine coronavirus HKU15, WIV1-CoV, SHCO14-CoV, bat-SL-CoVZC45, bat-SLCoVZXC21, SARS-CoVGZ02, BtKY72, WIV16, Rs4231, Rs7327, Rs9401, BtRs-BetaCoV/YN2018R, BtRs-BetaCoV/YN2013, Anlong-112, Rf2092, BtRs-BetaCoV/YN2018C, As6526, Rs4247, BtRs-BetaCoV/GX2013, Yunnan2011, BtRl-BetaCoV/SC2018, Shannxi2011, BtRs-BetaCoV/HuB2013, Bat_CoV_279/2005, HKU3-12, HKU3-3, HKU3-7, Longquan-140, or RaTG13.

In some aspects, the coronavirus can be SARS-CoV, MERS-CoV, SARS-CoV-2, HCoV 229E, HCoV NL63, HCoV OC43, or HCoV HKU1.

In some aspects, the respiratory virus can be a coronavirus, an influenza A virus, an influenza B virus, a Parainfluenza virus, a respiratory syncytial virus (RSV), a severe acute respiratory coronavirus syndrome-CoV (SARS-CoV), a middle east respiratory syndrome coronavirus (MERS-CoV), a human coronavirus (hCoV) 229e, a human coronavirus NL63, a human coronavirus HKU1, an human coronavirus OC43, an adenovirus, a rhinovirus, an enterovirus, a SARS-CoV-2, or a combination thereof.

### Compositions

The compositions described herein can be formulated to include a therapeutically effective dose (or amount) of sulforaphane. Therapeutic administration encompasses prophylactic applications. Based on genetic testing and other prognostic methods, a physician in consultation with their patient can choose a prophylactic administration where the patient has a clinically determined predisposition or increased susceptibility (in some cases, a greatly increased susceptibility) to a type of coronavirus or respiratory virus.

The compositions described herein can be formulated in a variety of combinations. The particular combination of sulforaphane with one or more antivirals can vary according to many factors, for example, the particular the type and severity of the infection. In some aspects, the antiviral can be remdesivir.

The compositions described herein can be administered to the subject (e.g., a human patient) in an amount sufficient to delay, reduce, or preferably prevent the onset of infection or clinical disease. Accordingly, in some aspects, the patient is a human patient. In therapeutic applications, compositions can be administered to a subject (e.g., a human patient) already with or diagnosed with an infection or a disease associated with the infection in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a composition (e.g., a pharmaceutical composition) can be an amount that achieves a cure, but that outcome is only one among several that can be achieved. As noted, a therapeutically effective amount includes amounts that provide a treatment in which the onset or progression of the infection (e.g., replication of the coronavirus) is delayed, hindered, or prevented, or the infection or a symptom of the infection or disease associated with the infection is ameliorated. One or more of the symptoms can be less severe. Recovery can be accelerated in an individual who has been treated.

Disclosed herein, are methods of treating a subject with a coronavirus infection. The coronavirus can be any coronavirus. In some aspects, the coronavirus is SARS-CoV, MERS-CoV, SARS-CoV-2, HCoV 229E, HCoV NL63, HCoV OC43, HCoV HKU1. In some aspects, the subject has been diagnosed with a coronavirus infection or a disease associated with a coronavirus infection (e.g., COVID-19) prior to the administering step.

The compositions described herein can be formulated to include a therapeutically effective amount of sulforaphane alone or in combination with one or more antivirals (e.g., remdesivir). In some aspects, sulforaphane can be contained within a pharmaceutical formulation. In some aspects, the pharmaceutical formulation can be a unit dosage formulation. In some aspects, the antiviral therapeutic can be a cellular or gene therapy therapeutic, an immunomodulatory, an antibody or mixture of antibodies or an antiviral. In some aspects, the antiviral therapeutic is remdesivir (Veklury), Nafamostat. Avigan (favilavir), bamlanivimab, Olumiant and Baricinix (baricitinib), hydroxychloroquine/chloroquine, Casirivimab and imdevimab (formerly REGN-COV2), PTC299, Leronlimab (PRO 140), Bamlanivimab (LY-CoV555), Lenzilumab, Ivermectin, RLF-100 (aviptadil), Metformin (Glucophage, Glumetza, Riomet), AT-527, Actemra (tocilizumab), Niclocide (niclosamide), Convalescent plasma, Pepcid (famotidine), Kaletra (lopinavir-ritonavir), Remicade (infliximab), AZD7442, AZD7442, CT-P59, Heparin (UF and LMW), VIR-7831 (GSK4182136), JS016, Kevzara (sarilumab), SACCOVID (CD24Fc), Humira (adalimumab), COVI-GUARD (STI-1499), Dexamethasone (Dextenza, Ozurdex, others), PB1046, Galidesivir, Bucillamine, PF-00835321 (PF-07304814), Eliquis (Apixaban), Takhzyro (lanadelumab), Hydrocortisone, Ilaris (canakinumab), Colchicine (Mitigare, Colcrys), BLD-2660, Avigan (favilavir/avifavir), Rhu-pGSN (gelsolin), MK-4482, TXA127, LAM-002A (apilimod dimesylate), DNL758 (SAR443122), INOpulse, ABX464, AdMSCs, Losmapimod, Mavrilimumab, or Calquence (acalabrutinib). In some aspects, the antiviral therapeutic can be one or more the antiviral therapeutics that can be used in combination with sulforaphane is described in De Clercq E. and Li G., Clin Microbiol Rev. 2016 July; 29(3):695-747, which is hereby incorporated by reference for its teaching of antiviral therapeutics.

The therapeutically effective amount or dosage of sulforaphane, and remdesivir used in the methods as disclosed herein applied to mammals (e.g., humans) can be determined by one of ordinary skill in the art with consideration of individual differences in age, weight, sex, other drugs administered and the judgment of the attending clinician. Variations in the needed dosage may be expected. Variations in dosage levels can be adjusted using standard empirical routes for optimization. The particular dosage of a pharmaceutical composition to be administered to the patient will depend on a variety of considerations (e.g., the severity of the symptoms of the infection), the age and physical characteristics of the subject and other considerations known to those of ordinary skill in the art. Dosages can be established using clinical approaches known to one of ordinary skill in the art.

The duration of treatment with any composition provided herein can be any length of time from as short as one day to as long as the life span of the host (e.g., many years). For example, the sulforaphane can be administered daily (including multiple times in the same day); once a week (for, for example, 2 or more weeks to many months or years); once a month (for, for example, two to twelve months or for many years); or once a year for a period of 5 years, ten years, or longer. It is also noted that the frequency of treatment can be variable. For example, the present compositions can be administered once (or twice, three times, etc.) daily, weekly, monthly, or yearly. In some aspects, for example, the remdesivir can be administered to subjects with COVID-19 intravenously, 200 mg, as a single dose on day 1, followed by 100 mg once daily. It is also noted that the frequency of treatment can be variable, and depend on several factors including but not limited to oxygen requirements and need for ventilatory support. For example, the present compositions can be administered once (or twice, three times, etc.) daily, weekly, monthly, or yearly.

In some aspects, sulforaphane can be administered to the subject immediately after infection or any time within one day to 5 days after infection or at the earliest time after diagnosis of infection with the coronavirus.

In some aspects, sulforaphane can be administered to the subject as a primary antiviral therapy, adjunct antiviral therapy, or a co-antiviral therapy, or wherein the administration comprises separate administration or coadministration of sulforaphane with at least one other antiviral composition or with at least one other composition for treating one or more symptoms associated with said coronavirus infection. In some aspects, the at least one other antiviral composition can be remdesivir.

Dosages of sulforaphane can be in the range of 25 μmol to 200 μmol/day. In some aspects, the dosage of sulforaphane can be 25, 50, 75, 100, 125, 150, 175 or 200 μmol total or any amount in between. In some aspects, the therapeutically effective dose of sulforaphane may be less when combined with remdesivir. In some aspects, the administration of sulforaphane increases the efficacy of remdesivir. In some aspects, the sulforaphane total dose per day can be independently selected upon each occurrence from about 25 μmol to about 200 μmol

Dosages of remdesivir can be in the range of 100 to 200 mg total or any amount in between. In some aspects, 200 mg of remdesivir can be administered as a single dose. In some aspects, 100 mg of remdesivir can be administered as a single dose. Suitable treatment regimens using any of the dosages described herein include but are not limited to 200 mg of remdesivir can be administered as a single dose on day 1, followed by 100 mg once daily for 2 or more days. In some aspects, 200 mg of remdesivir can be administered as a single dose on day 1, followed by 100 mg once daily for 2 or more days. In some aspects, 200 mg of remdesivir can be administered as a single dose on day 1, followed by 100 mg once daily for 3 or more days. In some aspects, 200 mg of remdesivir can be administered as a single dose on day 1, followed by 100 mg once daily for 4 days. In some aspects, 200 mg of remdesivir can be administered as a single dose on day 1, followed by 100 mg once daily for up to 4 days (or more) or until the subject is discharged from the hospital. In some aspects, In some aspects, 200 mg of remdesivir can be administered as a single dose on day 1, followed by 100 mg once daily up to 5, 6, 7, 8, 9 or 10 days. In some aspects, 200 mg of remdesivir can be administered as a single dose on day 1, followed by 100 mg once daily up to 10 days in patients without substantial clinical improvement at day 5 (Beigel 2020; FDA 2020a; NIH 2020). In some aspects, remdesivir can be administered in combination with dexamethasone. In some aspects, the therapeutically effective dose of remdesivir may be less when combined with sulforaphane.

The total effective amount of the compositions as disclosed herein can be administered to a subject as a single dose, either as a bolus or by infusion over a relatively short period of time, or can be administered using a fractionated treatment protocol in which multiple doses are administered over a more prolonged period of time. Alternatively, continuous intravenous infusions sufficient to maintain therapeutically effective concentrations in the blood are also within the scope of the present disclosure. In some aspects, the sulforaphane can be administered by a different route than remdesivir. In some aspects, sulforaphane and remdesivir can be co-formulated.

In some aspects, the therapeutically effective dose of sulforaphane and remdesivir can be in a ratio of 1:01 to 1:10. In some aspects, the therapeutically effective doses of sulforaphane and remdesivir can be administered in synergistic combination.

The compositions described herein can be administered in conjunction with other therapeutic modalities to a subject in need of therapy. The present compounds can be given to prior to, simultaneously with or after treatment with other agents or regimes. For example, sulforaphane alone or with any of the antivirals disclosed herein can be administered in conjunction with standard therapies used to treat a coronavirus or a respiratory virus. In some aspects, any of the compounds or compositions described herein can be administered or used together with an anti-inflammatory agent. In some aspects, any of the compounds or compositions described herein can be administered or used together with an immunomodulatory agent. In some aspects, the immunomodulatory agent is not a primary anti-inflammatory agent, and includes but is not limited to statins, estrogen therapy, and antibody therapies.

Any of the compounds or compositions described herein can be administered as a term “combination.” It is to be understood that, for example, sulforaphane can be provided to the subject in need, either prior to administration of remdesivir, concomitant with administration of remdesivir or any combination thereof (co-administration) or shortly thereafter.

Sulforaphane has antiviral activity through NRF-2 independent and NRF-2 dependent mechanisms. Other NRF2 agonists that have been tested for SARS-CoV-2, for example, 4-octyl-itaconate (4-OI) (Olagnier, D., et al. Nature Communications 11, 4938 (2020)). The antiviral activity of 4-OI did not overlap with any known antiviral mechanisms, suggesting it acts via an alternative pathway. In some aspects, sulforaphane has an additive or synergistic effect with one or more drug classes or antivirals.

In some aspects, in any of the methods disclosed herein, sulforaphane can be administered to a subject with one or more therapeutically effective doses of a polymerase inhibitor. In some aspects, the polymerase inhibitor can be molnupiravir, 4′-fluorouridine, favipiravir, or remdesivir. In some aspects, the therapeutically effective doses of sulforaphane and the polymerase inhibitor can be administered in an additive or synergistic combination.

In some aspects, in any of the methods disclosed herein, sulforaphane can be administered to a subject with one or more therapeutically effective doses of a protease inhibitor. In some aspects, the protease inhibitor can be nirmatrelvir, ritonavir, a combination of nirmatrevlir and ritonavir, or GC-376. In some aspects, the therapeutically effective doses of sulforaphane and the protease inhibitor can be administered in an additive or synergistic combination.

In some aspects, in any of the methods disclosed herein, sulforaphane can be administered to a subject with one or more therapeutically effective doses of a helicase inhibitor. In some aspects, the helicase inhibitor can be cepharanthine, cefoperazone, dihydroergotamine, cefpiramide, ergoloid, ergotamine, netupitant, Dpnh (NADH), lifitegrast, nilotinib, tubocurarin, lumacraftor, emend, irinotecan, enjuvia, zelboraf, cromolyn, diosmin, Risperdal, or differin. In some aspects, the therapeutically effective doses of sulforaphane and the helicase inhibitor can be administered in an additive or synergistic combination.

In some aspects, in any of the methods disclosed herein, sulforaphane can be administered to a subject with one or more therapeutically effective doses of an inhibitor of host proteins supporting viral replication. In some aspects, the inhibitor of host proteins supporting viral replication can be plitidepsin. In some aspects, the therapeutically effective doses of sulforaphane and the inhibitor of host proteins supporting viral replication can be administered in an additive or synergistic combination.

In some aspects, in any of the methods disclosed herein, sulforaphane can be administered to a subject with one or more therapeutically effective doses of a non-vaccine biologic. In some aspects, the non-vaccine biologic can be convalescent plasma, actemra, a monoclonal antibody specific for a viral protein. In some aspects, the therapeutically effective doses of sulforaphane and the non-vaccine biologic can be administered in an additive or synergistic combination.

In some aspects, in any of the methods disclosed herein, sulforaphane can be administered to a subject with one or more therapeutically effective doses of an inhibitor of viral attachment and entry. In some aspects, the inhibitor of viral attachment and entry can be human recombinant soluble angiotensin converting enzyme—2 (ACE2) or camostate mesylate and analogs thereof. In some aspects, the therapeutically effective doses of sulforaphane and the inhibitor of viral attachment and entry can be administered in an additive or synergistic combination.

In some aspects, in any of the methods disclosed herein, sulforaphane can be administered to a subject with one or more therapeutically effective doses of a selective serotonin reuptake inhibitor. In some aspects, the selective serotonin reuptake inhibitor can be fluvoxamine. In some aspects, the therapeutically effective doses of sulforaphane and the selective serotonin reuptake inhibitor can be administered in an additive or synergistic combination.

In some aspects, in any of the methods disclosed herein, sulforaphane can be administered to a subject with one or more therapeutically effective doses of one or more polymerase inhibitors, a protease inhibitors, helicase inhibitors, inhibitors of host proteins supporting viral replication, non-vaccine biologics, inhibitors of viral attachment and entry, or selective serotonin reuptake inhibitors. In some aspects, the therapeutically effective doses of sulforaphane and the one or more polymerase inhibitors, a protease inhibitors, helicase inhibitors, inhibitors of host proteins supporting viral replication, non-vaccine biologics, inhibitors of viral attachment and entry, or selective serotonin reuptake inhibitors can be administered in an additive or synergistic combination.

Pharmaceutical Compositions

Disclosed herein, are pharmaceutical compositions, comprising one or more of the therapeutic compositions disclosed herein. Disclosed herein, are pharmaceutical compositions, comprising sulforaphane (e.g., a therapeutically effective dose) and a pharmaceutical acceptable carrier, diluent or excipient as described herein. Disclosed herein, are pharmaceutical compositions, comprising sulforaphane (e.g., a therapeutically effective dose) and a pharmaceutical acceptable carrier, diluent or excipient as described herein and one or more polymerase inhibitors, a protease inhibitors, helicase inhibitors, inhibitors of host proteins supporting viral replication, non-vaccine biologics, inhibitors of viral attachment and entry, or selective serotonin reuptake inhibitors. In some aspects, sulforaphane can be formulated for oral or parenteral administration. In some aspects, the parenteral administration is intravenous, subcutaneous, intramuscular or direct injection. The compositions can be formulated for administration by any of a variety of routes of administration, and can include one or more physiologically acceptable excipients, which can vary depending on the route of administration. As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed.

The compositions can be administered directly to a subject. Generally, the compositions can be suspended in a pharmaceutically acceptable carrier (e.g., physiological saline or a buffered saline solution) to facilitate their delivery. Encapsulation of the compositions in a suitable delivery vehicle (e.g., polymeric microparticles or implantable devices) may increase the efficiency of delivery.

The compositions can be formulated in various ways for parenteral or nonparenteral administration. Where suitable, oral formulations can take the form of tablets, pills, capsules, or powders, which may be enterically coated or otherwise protected. Sustained release formulations, suspensions, elixirs, aerosols, and the like can also be used.

Pharmaceutically acceptable carriers and excipients can be incorporated (e.g., water, saline, aqueous dextrose, and glycols, oils (including those of petroleum, animal, vegetable or synthetic origin), starch, cellulose, talc, glucose, lactose, sucrose, gelatin, malt, rice, flour, chalk, silica gel, magnesium stearate, sodium stearate, glycerol monosterate, sodium chloride, dried skim milk, glycerol, propylene glycol, ethanol, and the like). The compositions may be subjected to conventional pharmaceutical expedients such as sterilization and may contain conventional pharmaceutical additives such as preservatives, stabilizing agents, wetting or emulsifying agents, salts for adjusting osmotic pressure, buffers, and the like. Suitable pharmaceutical carriers and their formulations are described in “Remington's Pharmaceutical Sciences” by E. W. Martin, which is herein incorporated by reference. Such compositions will, in any event, contain an effective amount of the compositions together with a suitable amount of carrier so as to prepare the proper dosage form for proper administration to the patient.

The pharmaceutical compositions as disclosed herein can be prepared for oral or parenteral administration. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation can also be used. Thus, compositions can be prepared for parenteral administration that includes sulforaphane or remdesivir dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like).

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules.

In some aspects, a pharmaceutical composition comprises sulforaphane. In some aspects, a pharmaceutical composition comprises sulforaphane and optionally, a pharmaceutical acceptable carrier, diluent or excipient. In some aspects, a pharmaceutical composition comprises sulforaphane and remdesivir, and optionally, a pharmaceutical acceptable carrier, diluent or excipient. Disclosed herein, are pharmaceutical compositions, comprising sulforaphane (e.g., a therapeutically effective dose) and one or more polymerase inhibitors, a protease inhibitors, helicase inhibitors, inhibitors of host proteins supporting viral replication, non-vaccine biologics, inhibitors of viral attachment and entry, or selective serotonin reuptake inhibitors and optionally, a pharmaceutical acceptable carrier, diluent or excipient.

In some aspects, the pharmaceutical composition can be formulated for oral or intravenous administration. In some aspects, the compositions described herein can be formulated for buccal, enteral, intramuscular, subdermal, sublingual, peroral, oral administration, or a combination thereof.

Articles of Manufacture

The compositions described herein can be packaged in a suitable container labeled, for example, for use as a therapy to treat, inhibit or prevent a coronavirus infection, inhibit replication of a coronavirus or any of the methods disclosed herein.

Disclosed herein are kits comprising one or more therapeutically effective doses of sulforaphane for inhibiting, treating or preventing a coronavirus infection in a subject. Disclosed herein are kits comprising one or more therapeutically effective doses of sulforaphane and one or more therapeutically effective doses of at least one other antiviral for inhibiting, treating or preventing a coronavirus infection in a subject. Disclosed herein are kits for use in treating a subject suffering from a coronavirus infection. In some aspects, the kit comprises: sulforaphane; and at least one other antiviral.

Disclosed herein are kits for use in preventing or inhibiting a coronavirus infection in a subject. Disclosed herein are kits for use in inhibiting replication of a coronavirus infection in a subject. In some aspects, the kits comprise: sulforaphane; and remdesivir. In some aspects, the kits can further comprise at least one pharmaceutically acceptable carrier, diluent or excipient. In some aspects, the kits can further comprise at least one polymerase inhibitor, a protease inhibitor, helicase inhibitor, inhibitor of host proteins supporting viral replication, non-vaccine biologic, inhibitor of viral attachment and entry, or selective serotonin reuptake inhibitor and optionally, a pharmaceutical acceptable carrier, diluent or excipient. In some aspects, the kits can comprise: (a) sulforaphane; and (b) molnupiravir, 4′-fluorouridine, favipiravir, remdesivir, nirmatrelvir, ritonavir, a combination of nirmatrevlir and ritonavir, GC-376, cepharanthine, cefoperazone, dihydroergotamine, cefpiramide, ergoloid, ergotamine, netupitant, Dpnh (NADH), lifitegrast, nilotinib, tubocurarin, lumacraftor, emend, irinotecan, enjuvia, zelboraf, cromolyn, diosmin, Risperdal, differin, plitidepsin, convalescent plasma, actemra, recombinant soluble ACE2, camostate mesylate and analogs thereof, or fluvoxamine.

In some aspects, the kits can further comprise instructions for using sulforaphane in treating a coronavirus infection. Accordingly, packaged products (e.g., sterile containers containing the composition described herein and packaged for storage, shipment, or sale at concentrated or ready-to-use concentrations) and kits, including at least sulforaphane as described herein and instructions for use, are also within the scope of the disclosure. A product can include a container (e.g., a vial, jar, bottle, bag, or the like) containing the composition described herein. In addition, an article of manufacture further may include, for example, packaging materials, instructions for use, syringes, buffers or other control reagents for treating or monitoring the condition for which prophylaxis or treatment is required. The product may also include a legend (e.g., a printed label or insert or other medium describing the product's use (e.g., an audio- or videotape)). The legend can be associated with the container (e.g., affixed to the container) and can describe the manner in which the compound therein should be administered (e.g., the frequency and route of administration), indications therefor, and other uses. The compounds can be ready for administration (e.g., present in dose-appropriate units), and may include a pharmaceutically acceptable adjuvant, carrier or other diluent. Alternatively, the compounds can be provided in a concentrated form with a diluent and instructions for dilution. In some aspects, sulforaphane and remdesivir can be co-packaged. In some aspects, sulforaphane and one or more polymerase inhibitors, a protease inhibitors, helicase inhibitors, inhibitors of host proteins supporting viral replication, non-vaccine biologics, inhibitors of viral attachment and entry, or selective serotonin reuptake inhibitors and optionally, a pharmaceutical acceptable carrier, diluent or excipient can be co-packaged.

### EXAMPLES

### Example 1: Sulforaphane Exhibits Antiviral Activity Against Seasonal HCoV-OC43 and Pandemic SARS-CoV-2 Coronaviruses

Sulforaphane (SFN) was investigated for efficacy against coronaviruses. The results show that SFN inhibits in vitro HCoV-OC43 and SARS-CoV-2 infections of mammalian host cells and appears to have a synergistic interaction with remdesivir. In addition, SFN reduces viral load and pulmonary pathology in a mouse model of SARS-CoV-2 infection. The results suggest that its use could be a rapidly applicable strategy for the prevention and treatment of COVID-19 as well as seasonal coronavirus infections.

Results. To evaluate the protective effect of sulforaphane, cells were exposed to the test drug for 1 to 3 hours before inoculation with coronaviruses. In this near-simultaneous drug-infection scenario, sulforaphane effectively inhibited both HCoV-OC43 and SARS-CoV-2-Wuhan-Hu-1 virus-associated cell death in non-human primate Vero C1008 cells in a dose-dependent manner revealing comparable median inhibitory concentrations (IC₅₀=10 μM; 12 μM, respectively), and virus selectivity (T₅₀=7; 7, respectively). When the same assay was performed using human diploid fibroblasts, MRC-5, host cells, sulforaphane treatment of HCoV-OC43 infection produced similar results (IC₅₀=18 μM, TI₅₀=5). Sulforaphane cytotoxicity was also dose-dependent; the mean cytotoxic dose, TD₅₀, remained within the range of 73-89 μM. Additionally, viral RNA from SARS-CoV-2 infected human intestinal Caco-2 cells treated with SFN was quantified. A dose-dependent reduction was observed with an IC50 of 2.4 μM.

SFN was tested for activity against two reference strains and two clinical strains of SARS-CoV-2. SFN inhibited both reference strains, Wuhan-Hu-1 and USA-WA1/2020, with comparable efficacy, IC₅₀=15-17 μM, as that reported above. Sulforaphane also moderately inhibited the two 614G+ clinical strains, MD and DC, with comparable efficacy with IC₅₀ concentrations (48 and 51 μM, respectively).

Next, it was investigated whether sulforaphane could affect an established virus infection. The results show that sulforaphane effectively inhibited both HCoV-OC43 and SARS-CoV-2-Wuhan-Hu-1 infection that had been allowed to replicate for 24 hours before addition of drug. Again the IC₅₀ for both viruses was in the lower micromolar range, 18 μM and 13 μM, respectively. Interestingly, the readouts for the two different assay formats with SARS-CoV-2-Wuhan-Hu-1 show that the selectivity of sulforaphane is identical whether the drug is added just before or 24 hours after the virus inoculation.

It was also determined whether a single application of sulforaphane could provide protection from the cytopathic effects (CPE) of subsequent viral infection. The results also show that SFN pretreatment of Vero C1008 host cells resulted in measurable inhibition of HCoV-OC43 CPE with an IC₅₀=21 μM and TI₅₀=4.

Finally, the potential synergistic effects of sulforaphane combined with the anti-viral drug remdesivir, an inhibitor of viral RNA-dependent, RNA polymerase recently reported to shorten the time to recovery in adults who were hospitalized with Covid-19 was examined. The results show that remdesivir effectively inhibits in vitro replication of four strains of SARS-CoV-2 (IC₅₀=4-9 μM) as well as HCoV-OC43 albeit at higher a concentration (IC₅₀=22 μM). In two-drug combination assays, SFN and remdesivir interacted synergistically at several combination ratios to inhibit replication of both HCoV-OC43 and SARS-CoV-2-Wuhan-Hu-1.

To evaluate the therapeutic effect of sulforaphane in vivo, K18-hACE2 transgenic male mice were inoculated intranasally with 8.4×10⁵ tissue culture infectious dose 50 (TCID₅₀) of SARS-CoV-2/USA/WI1/2020. Sulforaphane was administered daily via oral gavage to a subgroup of infected animals starting one day prior to viral inoculation. A marked weight loss was observed in the infected animals starting at four days post inoculation. By day 6 post inoculation, sulforaphane treated mice lost significantly less weight compared to controls (P=0.004). As a measure of lung injury, the protein concentration in the bronchoalveolar lavage (BAL) was also lower in the sulforaphane treated mice compared to controls (P=0.01). The viral burden measured in the BAL was significantly lower in treated animals compared to controls (P=0.03). However, no statistically significant differences were observed in the lung viral load. Analysis of hematoxylin and eosin-stained lung sections from these animals showed an inflammatory process after SARS-CoV-2 infection. Sulforaphane treated mice had a lower degree of pulmonary pathology with less alveolar infiltrates compared to infected untreated mice. SARS-CoV-2 spike protein was present in both treated and untreated animals, compromising a smaller area of the lungs of sulforaphane-treated mice compared to those not treated.

Limited differences in overall immune cell composition in the spleen or lungs between treated and untreated mice were observed. Sulforaphane treated mice demonstrated significantly higher expansion of B cells in the spleen and Ly6G+ neutrophils in the lung. However, both alveolar and interstitial macrophages demonstrated immunological rewiring in response to sulforaphane treatment. Following treatment, macrophages expressed lower levels of programmed death ligand 1 (PD-L1), a potent suppressor of T cell responses through interaction with its ligand, PD-1. Further, sulforaphane treatment reduced CCR2 expression on alveolar macrophages. CCR2 is involved in egress from the bone marrow and infiltration of monocytes and macrophages into inflamed tissue. CCR2 has been implicated in promoting pulmonary fibrosis (Moore, B. B., et al., Journal of Immunology, 167(8):4368-4377), and inhibiting CCR2 signaling has also been shown to skew macrophages towards an inflammatory “M1” phenotype (Deci, M. B., et al., Molecular Pharmaceutics, 15(7):2721-2731). In contrast, lung-resident macrophages expressed increased CD86, a co-stimulatory molecule that plays a role in induction of robust T cell responses. Together, these data indicate that sulforaphane reprograms lung-resident macrophages during SARS-CoV-2 infection towards a stimulatory and non-immunosupressive phenotype. In response to sulforaphane treatment, macrophages also produced increased IL-10 and TGFβ. Both of these pleiotropic cytokines can be immunosuppressive in chronic conditions, however, they may be beneficial in preventing overactivation of the immune system and in skewing lung T cells towards a tissue resident phenotype in an acute setting (Thompson, E. A., et al. Cell Reports, 28(5):1127-1135). In accordance with a more pro-inflammatory innate compartment, there were increased frequency of B cells, cytotoxic NK cells, and CD8 T cells following sulforaphane treatment. When evaluated immediately ex vivo without additional stimulation, CD8 T cells from sulforaphane treatment mice showed signs of increased activation via upregulation of the proliferation marker Ki67 and activation marker MHCII, without signs of exhaustion, such as PD1. Together, these data support a role for sulforaphane in immunological rewiring during SARS-CoV-2 infection that promotes pro-inflammatory innate compartment and activated cytotoxic CD8 T cells to better control viral replication.

Discussion. The pathogenesis of many viral infections is associated with increased production of reactive oxygen species (ROS) which lead to cell death (Lee, C., Oxidative Medicine and Cellular Longevity, 2018. 2018: p. 6208067). One of the important pathways involved in response to this oxidative stress is regulated by the nuclear factor (erythroid-derived 2)-like 2 (NRF2). NRF2 is a cap‘n’collar transcription (CNC) transcription factor which remains in an inactive state in the cytosol by association with its inhibitor protein Kelch-like ECH-associated protein 1 (KEAP1) (Hayes, J. D. and A. T. Dinkova-Kostova, Trends in biochemical sciences, 2014. 39(4): p. 199-218). In response to oxidative stress, KEAP1 is inactivated and NRF2 is released to induce NRF2-responsive genes. In general, the genes under the control of NRF2 protect against stress-induced cell death and NRF2.

SARS-CoV-2 uses the angiotensin-converting enzyme 2(ACE2) receptor as one of the primary mechanisms of cell entry. NRF2 deficiency is known to upregulate ACE2, whereas its activator oltipraz reduces ACE2 levels, suggesting that NRF2 activation might reduce the availability of ACE2 for SARS-CoV-2 entry into the cell (Zhao, S., et al., Endocrinology, 2018. 159(2): p. 836-852). Increased NRF2 activity also inhibits IL-6 and IL-1β gene expression (Kobayashi, E. H., et al., Nature Communications, 2016. 7(1): p. 11624), which are known to play an important role in promoting the hyperactive immune response in severe COVID-19 patients (Moore, J. B. and C. H. June, Science, 2020. 368(6490): p. 473-474). Conversely, NRF2 activity is dysregulated in disease states that have been associated with increased severity of COVID-19 (e.g., diabetes) (Rabbani, P. S., et al., Diabetes, 2019. 68(1): p. 141-155). Similarly, NRF2 activity declines in older patients which are also more susceptible to severe COVID-19 (Schmidlin, C. J., et al., Free Radical Biology and Medicine, 2019. 134: p. 702-707). Recent reports suggest that NRF2-dependent genes are suppressed in biopsies from COVID-19 patients (Olagnier, D., et al., Nature Communications, 2020. 11(1): p. 4938). Similarly, treatment of cells with NRF2 agonists 4-octyl-itaconate and dimethyl fumarate inhibited replication of SARS-CoV-2 in vitro (Olagnier, D., et al., Nature Communications, 2020. 11(1): p. 4938). The suppression of the NRF-2 pathway, determined as the expression of NRF2-inducible proteins Heme Oxygenase 1 (HO-1) and NAD(P)H quinone oxydoreductase 1 (NqO1), was previously observed in SARS-CoV2 infected Vero hTMPRSS2 cells (Olagnier, D., et al., Nature Communications, 2020. 11(1): p. 4938).

Sulforaphane was tested for in vitro activity against SARS-CoV2. One limitation to this approach is the need for BSL-3 level containment facilities for the handling of SARS-CoV2. Thus, the seasonal beta-Coronavirus OC-43, which can be handled in widely available BSL-2 facilities, was used for initial testing. When sulforaphane was shown to inhibit the replication of HCoV-OC43 it was subsequently tested for in vitro and in vivo activity against SARS-CoV2 in BSL-3 facilities.

Conclusion. The results show that sulforaphane has the ability to inhibit the in vitro and in vivo replication of SARS-CoV2 and decrease the consequences of infection in the animal model when administered prior to infection.

Methods. Drugs: L-Sulforaphane, 10 mg/mL in ethanol (56 mM), was obtained from Cayman Chemical (Ann Arbor, MI). D,L-Sulforaphane was obtained from Millipore Sigma (St. Louis, MO); stock solution of 5 mM was prepared in DMSO. Remdesivir was obtained from MedChemExpress or Cayman Chemical and stock solutions, 5 or 20 mM, respectively, were prepared in DMSO. Drug stock solutions were stored at −25° C.

Cells and viruses: Cells were obtained from the American Type Culture Collection (ATCC, Manassas, VA, USA). HCT-8 [HRT-18] (ATCC CCL-244) and Vero C1008 [Vero 76, clone E6, Vero E6] (ATCC CRL-1586) cells were used for growing virus and determining virus stock titers. Vero C1008 cells, MRC-5 (ATCC CCL-171) cells, and Caco-2 (ATCC HTB-37) cells were used as host cells in antiviral assays. HCT-8 cells were grown in RPMI-1640 medium supplemented with 10% fetal bovine serum (FBS) (MilliporeSigma, St. Louis, MO, USA), L-glutamine, penicillin-streptomycin, and sodium pyruvate. Vero C1008 and MRC-5 cells were grown in EMEM with 10% FBS, L-glutamine, and penicillin-streptomycin at 37° C. with 5% CO₂. Caco-2 cells were grown in Minimum Essential Media supplemented with 10% FBS, 1× sodium pyruvate and penicillin-streptomycin at 37° C. with 5% CO₂. Human coronavirus OC43 (HCoV-OC43) was purchased from ATCC (Betacoronavirus 1, ATCC VR-1558). SARS-CoV-2/Wuhan-1/2020 virus (U.S. Centers for Disease Control and Prevention) was provided. 2019-nCoV/USA-WA1/2020 was obtained through BEI Resources, National Institute of Allergy and Infectious Diseases (NIAID), National Institutes of Health (NIH). Two 614G+ clinical strains of SARS-CoV-2, SARS-CoV-2/USA/DCHP-7/2020 (DC) and SARS-CoV-2/USA/MDHP-20/2020 (MD) were isolated from patients. The virus stocks were stored at −80° C. and titers were determined by tissue culture infectious dose 50 (TCID₅₀) assay.

Cytopathic effect (CPE) inhibition assay: A colorimetric assay that interrogates both antiviral and anti-host cell activities to evaluate compounds was used (Revu O, et al., Chemistry Select 2016. 1(18):5895-5899). This assay is predicated upon the virus's ability to cause a cytopathic effect (CPE), measured in TCID₅₀. Host cells, 7.5-10×10³ in virus growth medium (VGM; Dulbecco's Modified Eagle Medium without phenol red supplemented with 3% FBS), were plated in clear 96-well half-area tissue cultures plates or white, clear-bottom 96 well plates, 24 hours prior to the assay. On the day of the assay, working solutions of drugs (0.1-1 mM) were made by dilution of drug stocks in VGM. For one-drug analysis assays, 50 μL of the drug working solution was added to each well in the first column of cells and then drugs were serially diluted across the plate by dilutions of 0.5 log₁₀. The default drug test range was 320-0.032 μM. Drug-exposed cells were incubated for 1-24 hours at 35° C. (HCoV-OC43)—37° C. (SARS-CoV-2), after which time 32-50 TCID₅₀ of virus suspended in VGM or VGM alone was added to cells. Test plates had virus control wells (virus+/drug−), drug control wells (virus−/drug+), and cell control wells (virus−/drug−). After 3-4 days incubation at 35° C.-37° C. /5% CO₂, the cell viability was assessed using Celltiter 96®AQᵤₑₒᵤₛ One Solution (Promega Corp, WI, USA) or CellTiter-Glo® One Solution Assay system (Promega) following manufacturer protocols. Color reactions were read at 490-650 nm absorbance in a Filtermax F5 microplate reader (Molecular Devices, CA, USA) using SoftMax Pro 6.5 software. Luminescence readouts were obtained in a FLUOstar Omega plate reader (BMG Labtech, Ortenberg, Germany). For two-drug combination assays, one test drug was serially diluted across the plate (left to right) as described herein; the second drug was serially diluted down the plate (top to bottom). The starting concentration of the first drug was adjusted to allow for dilution with the second drug. For interrogation of a drug's ability to affect an established viral infection, host cells were infected with virus and allowed to incubate 24 hours. After this time the VGM in the wells was removed, the cells rinsed with Dulbecco's phosphate buffered saline, and then drug dilutions in the ranges mentioned above, or VGM alone, were added to appropriate wells. Cell viability reagent was added at 3-4 days post infection. For examination of a drug's ability to prevent an infection by pretreatment of the host cells, the test drug was serially diluted across the plate as described herein. After 24 hours incubation, the drug was removed by aspiration, the cells were rinsed once with warm Hanks Balanced Salt Solution, and then 32 TCID₅₀ of virus was added to appropriate wells. Cell viability reagent was added at 4 days post infection.

In vitro data analysis: Calcusyn software (Biosoft, Cambridge, UK) was used to calculate the median inhibitory concentration (IC₅₀), median cytotoxic dose (TD₅₀), and to generate median effect plots and dose response curves. The therapeutic index (TI), a measure of antiviral selectivity, was calculated by the formula TI=TD₅₀/IC₅₀. Combination Indices (CI) for two-drug combination assays were calculated by Calcusyn software. Isobolograms to depict synergistic, additive, and antagonistic combinations were generated by the software.

vRNA determination: Zymo Quick-RNA Viral 96 Kit (Zymo Research) was used to isolate RNA from cell supematants according to the manufacturer's protocol. cDNA synthesis was performed using qScript cDNA Supermix containing randomhexamers and oligo-dT primers following the manufacture's protocol (Quanta Biosciences). Real-time PCR was performed in technical triplicate for each sample using TaqMan Fast Advanced Master Mix (Applied Biosystems) on a StepOne Plus Real Time PCR machine (Applied Biosystems). Primers and probes are listed below. The cycling parameters were as follows: (i) 2 min at 50° C.; (ii) 2 min at 95° C.; and (iii) 45 cycles at 95° C. for 3s and 55° C. for 30 s. Molecular standard curves were generated using serial dilutions of a plasmid containing the complete SARS-CoV-2N gene (Integrated DNA Technologies, Catalog #10006625). SARS-CoV-2 RNA was detected using premixed forward (5′-TTACAAACATTGGCCGCAAA-3′; SEQ ID NO: 1) and reverse (5′-GCGCGACATTCCGAAGAA-3′; SEQ ID NO: 2) primers and probe (5′-FAM-ACAATITGCCCCCAGCGCTTCAG-BHQ1-3′; SEQ ID NO: 3) designed by the U.S. CDC and Prevention (CDC) as part of the 2019-nCoV CDC Research Use Only (RUO) kit (Integrated DNA Technologies, Catalog #10006713) to amplify a region of the SARS-CoV-2 nucleocapsid (N) gene.

Animals: Heterozygous K18-hACE c57BL/6J mice (strain: 2B6.Cg-Tg(K18-ACE2)2Prlmn/J) were obtained from The Jackson Laboratory and propagated at Johns Hopkins University School of Medicine. Animals were separately housed in groups and fed standard chow diets. Male mice, 6-8 weeks old were used for this study. A subgroup of animals received 30 mg/kg daily of SFN diluted in 5% ethanol and water via oral gavage. Treatment started one day prior to viral infection. Infected untreated and uninfected controls also received daily oral gavage with 5% ethanol and water. After induction of anesthesia with ketamine hydrochloride and xylazine, the animals received 8.4×10⁵ TCID₅₀ of SARS-CoV-2/USA/WI1/2020 intranasally. Weights were monitored daily, and the animals were sacrificed 6 days post-infection by isoflurane overdose, and the tissues were harvested. Tissues were perfused with PBS after serum collection via cardiac puncture and before tissue harvest. Broncheoalveolar lavage (BAL) was obtained by cannulating the trachea with a 20-gauge catheter. The right lung was lavaged twice (each aliquot 1 ml; calcium-free PBS); total returns averaged 1-1.5 ml/mouse. BAL was centrifuged at 600 g for 8 minutes at 4° C. The cell-free supematants were stored at −80° C. for total protein quantification using the BCA protein assay (Sigma).

Flow cytometry: Lungs were minced and incubated at 37° C. in an enzyme cocktail of RPMI containing 2.4 mg/ml collagenase I and 20 μg/ml DNase (Invitrogen), then mashed through a 70-μm nylon cell strainer (BD Falcon). The flow cytometry antibodies used for phenotypic and metabolic analysis can be found in FIG. 4. For analysis immediately ex vivo, cells were washed once in PBS and immediately stained for viability with Biolegend Live/Dead Zombie NIR Fixable Viability Dye and Fc Block for 10 min at room temperature. Cell surface staining was performed in 100 uL of 20% BD Horizon™ Brilliant Stain Buffer+ PBS with surface stain antibody cocktail for 20 min at room temperature. Cells were fixed and permeabilized with eBioscience™ FoxP3/Transcription Factor Staining kit 1× Fixation/Permeabilization reagent overnight at 4° C. Cells were washed with 1× Permeabilization/Wash buffer. Intracellular staining (ICS) was performed in 100 uL 1× Permeabilization/Wash buffer with ICS antibody cocktail for 45 min at room temperature. Cells were washed once with Permeabilization/Wash buffer then resuspended in Permeabilization/Wash buffer for acquisition by flow. Samples were run on a 3 laser Cytek Aurora spectral flow cytometer. FCS files were analyzed using Flowjo v10 (10.6.2.) software.

Histology and immunohistochemistry: After euthanasia, tissues were fixed on 10% neutral-buffered formalin. Tissues were embedded in paraffin and sections were stained with hematoxylin and eosin. Immunostaining was performed at the Oncology Tissue Services Core of Johns Hopkins University School of Medicine. Immunolabeling for the SARS-CoV-2 spike protein was performed on formalin-fixed, paraffin embedded sections on a Ventana Discovery Ultra autostainer (Roche Diagnostics). Briefly, following dewaxing and rehydration on board, epitope retrieval was performed using Ventana Ultra CC1 buffer (catalog #6414575001, Roche Diagnostics) at 96° C. for 64 minutes. Primary antibody, anti-Spike (1:200 dilution; catalog #GTX135356, Lot Number 43957, Genetex) was applied at 36° C. for 60 minutes. Primary antibodies were detected using an anti-rabbit HQ detection system (catalog #7017936001 and 7017812001, Roche Diagnostics) followed by Discovery Purple IHC detection kit (catalog #7053983001, Roche Diagnostics), counterstaining with Mayer's hematoxylin, dehydration and mounting.

### Example 2: Sulforaphane Exhibits Antiviral Activity Against Pandemic SARS-CoV-2 and Seasonal HCoV-OC43 Coronaviruses. In Vitro and in Mice

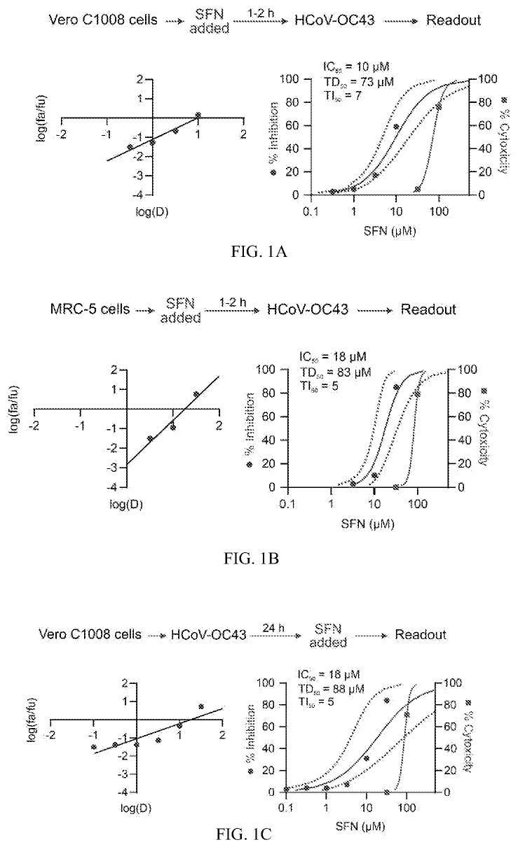

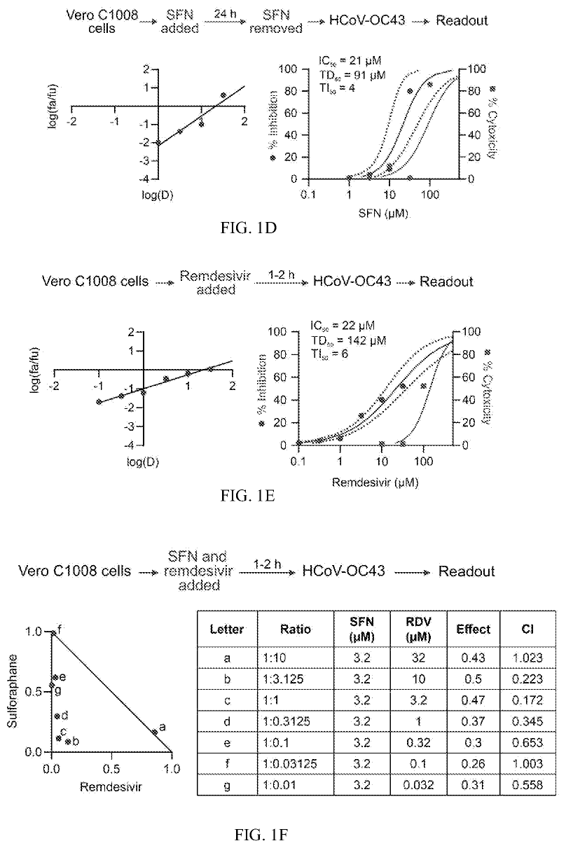

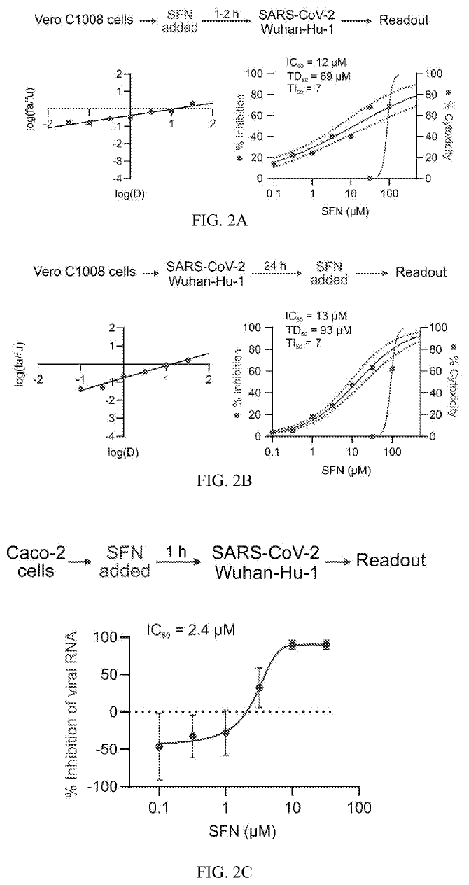

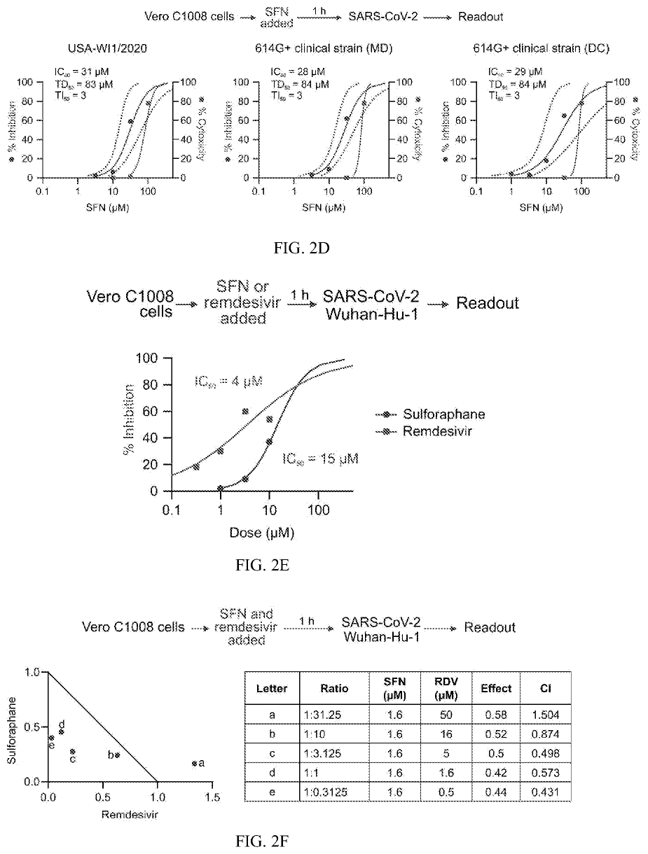

Antiviral effects of SFN against HCoV-OC43 and SARS-CoV-2. To evaluate the potential virus-inhibitory activity of SFN, cells were exposed in vitro to SFN for 1-2 hours before inoculation with coronaviruses. In this near-simultaneous drug-infection scenario, SFN effectively inhibited both HCoV-OC43 and SARS-CoV-2-Wuhan-Hu-1 virus-associated cell death in non-human primate Vero C1008 cells in a dose-dependent manner revealing comparable median inhibitory concentrations (IC₅₀=10 μM, 95% CI 4.7-20.4, and 12 μM, 95% CI 4.7-30, respectively), and virus selectivity [therapeutic index (TI)₅₀=7 and 7, respectively] (FIGS. 1A, 2A; Table 1). When the same assay was performed using human diploid fibroblasts, MRC-5, SFN treatment of HCoV-OC43 infection produced similar results (IC₅₀=18 μM, 95% CI 9.7-33.5, TI₅₀=5) (FIG. 1B). SFN cytotoxicity was also dose-dependent. The median cytotoxic dose (TD₅₀)remained within the range of 73-89 μM (FIGS. 1A-B, 2A). SARS-CoV-2-associated cytopathogenicity was not evaluated in human cells because viral infection did not result in measurable cell death. Instead, viral RNA was quantified from SARS-CoV-2 infected human intestinal Caco-2 cells treated with SFN. A dose-dependent reduction was observed with an IC₅₀ of 2.4 μM (FIG. 2C).

TABLE 1
Antiviral effects of SFN against HCoV-OC43.

| | Cell | Drug^{a} (range | Time of drug | Time of virus | IC₅₀ ^{b} | TD₅₀ ^{b} | |
|---|---|---|---|---|---|---|---|
| Panel | line | tested, μM) | addition | inoculation | (μM) | (μM) | TI ^{b} |
| A | Vero | SFN (320-0.032) | 1-2 h | 1-2 h | 10 | 73 | 7 |
| | C1008 | | before virus | after drug | | | |
| B | MRC-5 | SFN (320-0.032) | 1-2 h | 1-2 h | 18 | 83 | 5 |
| | | | before virus | after drug | | | |
| C | Vero | SFN (320-0.032) | 24 h after | 24 h | 18 | 88 | 5 |
| | C1008 | | virus | before drug | | | |
| D | Vero | SFN (100-0.01) | 24 h before | 24 h | 21 | 91 | 4 |
| | C1008 | | virus | after drug: | | | |
| | | | | drug washed | | | |
| | | | | out and then | | | |
| | | | | virus added | | | |
| E | Vero | RDV (100-0.01) | 1-2 h | 1-2 h | 22 | 142 | 6 |
| | C1008 | | before virus | after drug | | | |
| F | Vero | SFN (100-0.01) L to R^{c} | 1-2 h | 1-2 h | N/A^{d} | N/A^{d} | N/A^{d} |
| | C1008 | RDV (32-0.032) T to B | before virus | after drug | | | |

^{a}SFN, Sulforaphane; RDV, Remdesivir
^{b} IC₅₀, Median inhibitory concentration; TD₅₀, Median cytotoxic dose; TI, Therapeutic index
^{c}SFN diluted across plate, Left to Right (L to R); RDV diluted down plate, Top to Bottom (T to B)
_{d}N/A, Not applicable. See FIG. 1 and Results for Combination Index (CI) results

SFN was further evaluated for activity against a second reference strain of SARS-CoV-2 as well as two clinical strains that carry the spike D614G (614G+) substitution that is found in the majority of variants of concern currently in circulation (FIG. 2D) (Thou, B., et al. Nature 592, 122-127 (2021)). SFN inhibited USA-WA1/2020, (IC₅₀=31 JPM, 95% CI 14.7-66.4) and the two 614G+ clinical strains, USA/MDHP-20/2020 (MD) and USA/DCHP-7/2020 (DC) (IC₅₀=28 μM 95% CI 14.9-52.9, and 29 μM 95% CI 8.2-102.3, respectively), with comparable efficacy to that reported above for reference strain Wuhan-Hu-1.

It was next investigated whether SFN could affect an established virus infection. As shown in FIGS. 1C and 2B, SFN effectively inhibited both an HCoV-OC43 and a SARS-CoV-2-Wuhan-Hu-1 infection that had been allowed to replicate for 24 hours before addition of the drug. The IC₅₀ for both viruses was in the lower micromolar range, 18 μM (95% CI 4-84.1) and 13 μM (95% CI 8.6-20), respectively. Interestingly, these results show that the virus specific inhibitory activity, i.e., the TI, of SFN is similar whether the drug is added just before or 24 hours after virus inoculation (FIGS. 1A, 1C, 2A-B), demonstrating an effect on both extracellular entry and intracellular post-entry viral processes. It was also determined whether a single application of SFN could protect from the cytopathic effects (CPE) of subsequent viral infection lasting 4 days. As shown in FIG. 1D, SFN pretreatment of Vero C1008 host cells resulted in measurable inhibition of HCoV-OC43 CPE with an IC₅₀=21 μM (95% CI 9.3-49.3) and TI₅₀=4.

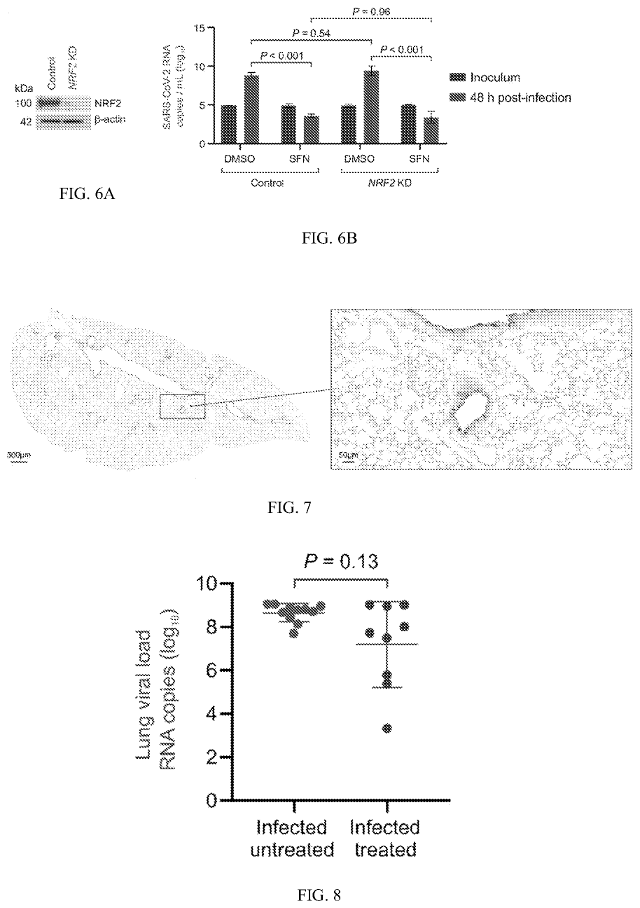

The synergistic effects of SFN combined with the anti-viral drug remdesivir, an inhibitor of viral RNA-dependent RNA polymerase reported to shorten the time to recovery in adults who were hospitalized with COVID-19 (Beigel, J. H., et al. N Engl J Med 383, 1813-1826 (2020)) was examined. As shown in FIG. 2E, remdesivir effectively inhibits in vitro replication of SARS-CoV-2 (IC₅₀=4 μM) as well as HCoV-OC43, albeit at a higher concentration (IC₅₀=22 μM) (FIG. 1E). In two-drug combination assays, SFN and remdesivir interacted synergistically at several combination ratios to inhibit replication of both HCoV-OC43 (FIG. 1F) and SARS-CoV-2-Wuhan-Hu-1 (FIG. 2F) at concentrations below the corresponding IC₅₀ for each drug. Finally, to evaluate the role of NRF2 in the antiviral activity of SFN, modified Caco-2 cells with decreased expression of NRF2 were infected with SARS-CoV-2 and treated with SFN. A similar reduction in SARS-CoV-2 viral load after SFN treatment in both control and NRF2 knockdown cells was observed, showing that the in vitro antiviral activity of SFN is likely to be mediated through an NRF2-independent pathway (FIG. 6).

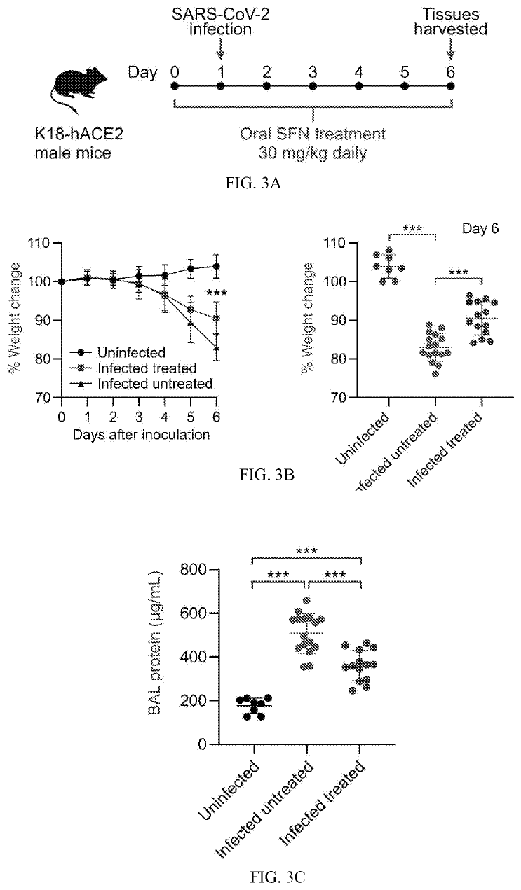

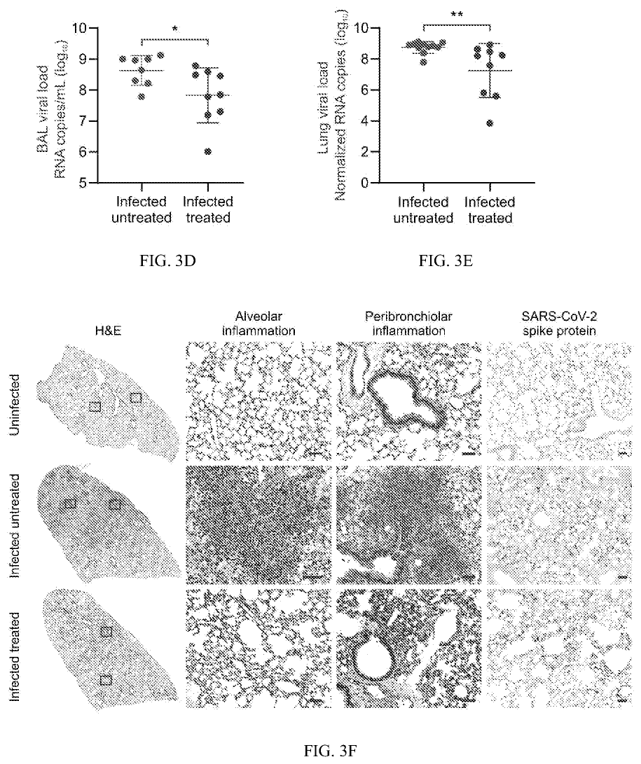

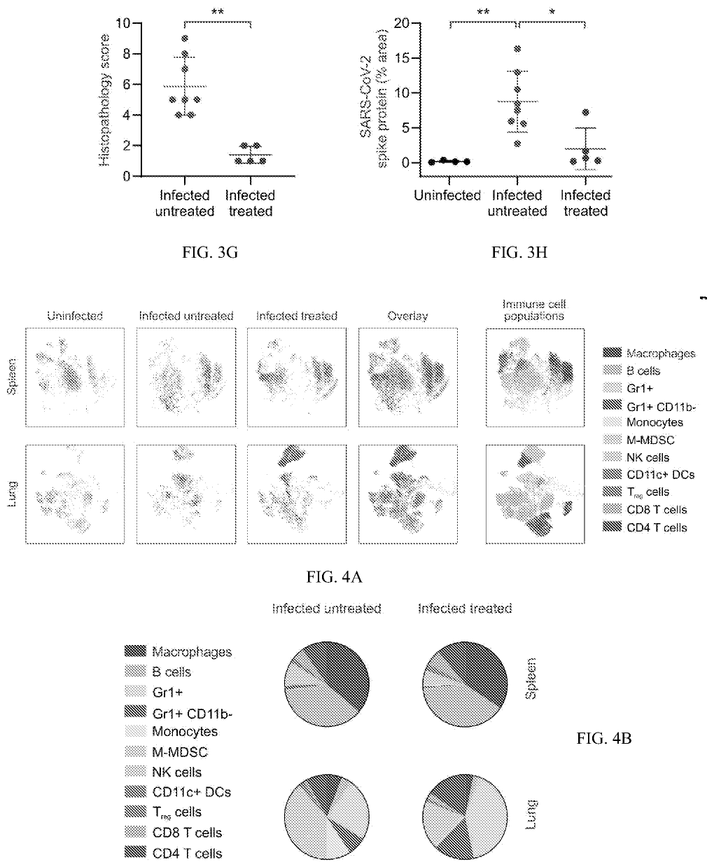

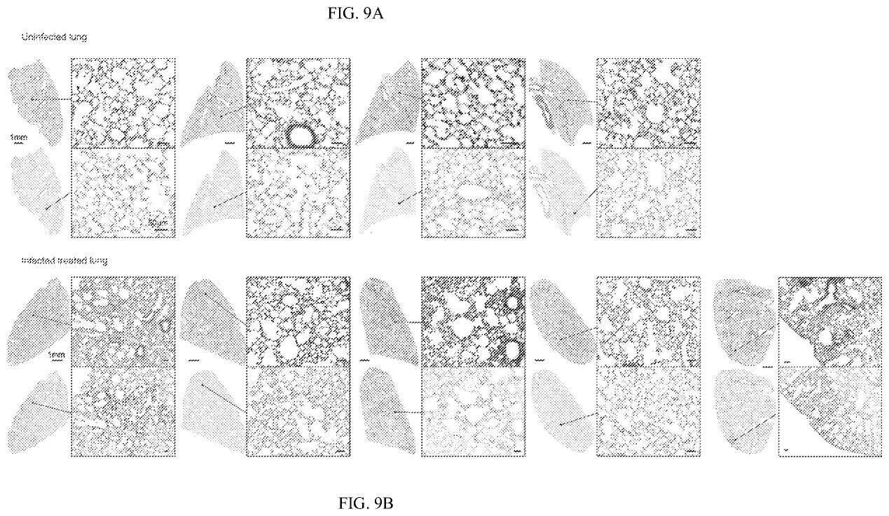

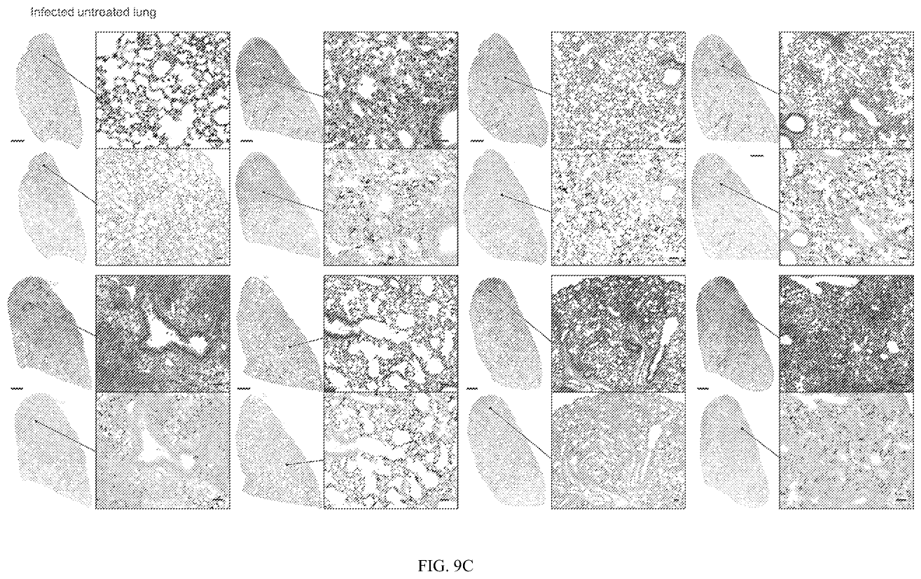

Effects of SFN treatment in SARS-CoV-2 infected mice. To evaluate the ability of SFN treatment to reduce viral titers and inflammation in vivo, K18-hACE2 transgenic male mice were inoculated intranasally with 8.4×10⁵ tissue culture infectious dose 50 (TCID₅₀) of SARS-CoV-2/USA/WI1/2020 (Zheng, J., et al. Nature 589, 603-607 (2021)). In K18-hACE2 mice, the human keratin 18 promoter directs expression of hACE2 to epithelia, allowing entry of SARS-CoV-2 into cells (FIG. 7). SFN was administered daily via oral gavage (dose of 30 mg/kg bodyweight) to a subgroup of infected animals starting one day prior to viral inoculation (FIG. 3A). A marked weight loss was observed in the infected animals starting at four days post inoculation. By day 6 post inoculation, SFN-treated mice lost significantly less weight compared to controls (FIG. 3B, P<0.0001). As a measure of lung injury, the protein concentration in the bronchoalveolar lavage (BAL) was significantly lower in the SFN-treated infected mice compared to untreated infected controls (FIG. 3C, P<0.0001), demonstrating a measure of protective effect of drug pretreatment. The viral burden measured in the alveolar fluid was also significantly lower in treated animals compared to untreated controls (FIG. 3D, P=0.04). Similarly, a 1.5 log reduction in viral lung titers was observed in SFN-treated mice compared to untreated controls when normalized to Pol2Ra (FIG. 3E, P=0.004). Data on pulmonary viral burden without normalization are presented in FIG. 8. Analysis of hematoxylin and eosin-stained lung sections from these animals showed an inflammatory process similar to what has been previously described for this model after SARS-CoV-2 infection (Zheng. J., et al. Nature 589, 603-607 (2021); Winkler, E. S., et al. Nat Immunol 21, 1327-1335 (2020); and Oladunni, F. S., et al. Lethality of SARS-CoV-2 infection in K18 human angiotensin-converting enzyme 2 transgenic mice. Nat Commun 11, 6122 (2020) (FIG. 3F). SFN-treated mice had a lower degree of pulmonary pathology with less alveolar and peribronchiolar inflammation compared to infected untreated mice (FIG. 9). Histopathology analysis showed a significant reduction of lung inflammation in SFN-treated mice (histopathology score of 1/16) over untreated controls (histopathology score of 6/16) (FIG. 3G, P=0.0008). Immunostaining for SARS-CoV-2 spike protein revealed a more widespread distribution in the lungs of infected untreated animals compared to a focal distribution in those of treated animals (FIGS. 3F, FIG. 9). Quantification of the SARS-CoV-2 spike protein immunostaining revealed that the lung area associated with the virus was 4.4× higher in the infected untreated animals compared to the SFN-treated mice (FIG. 3H, P=0.01).

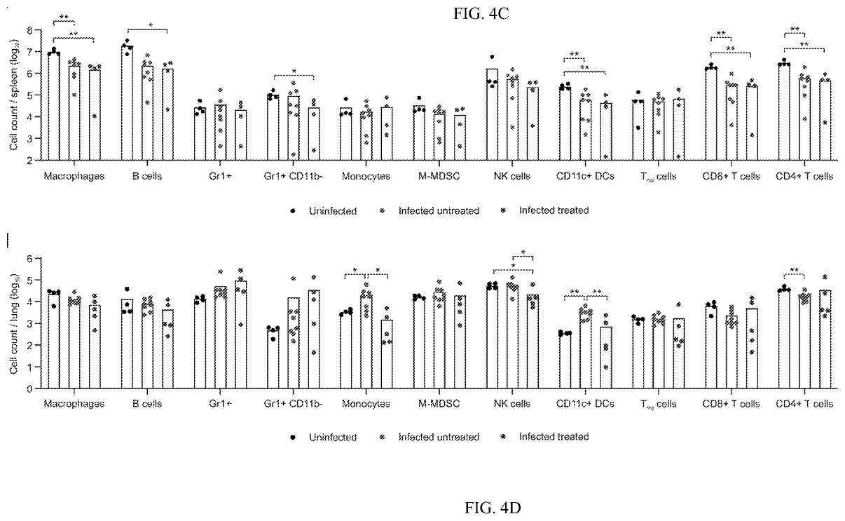

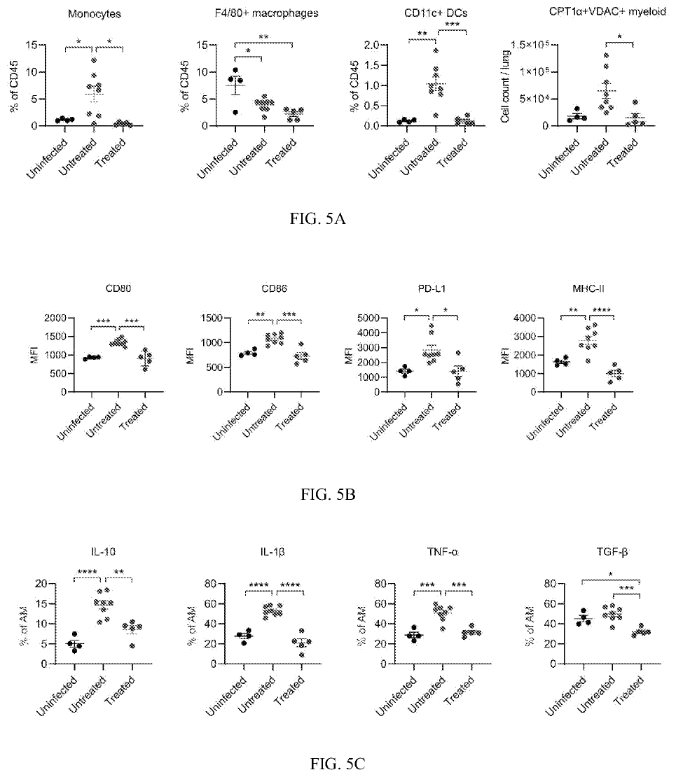

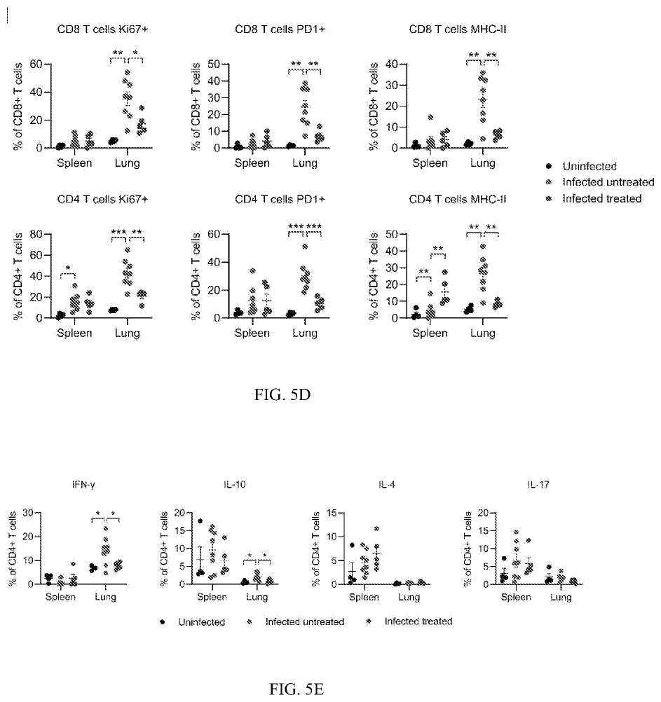

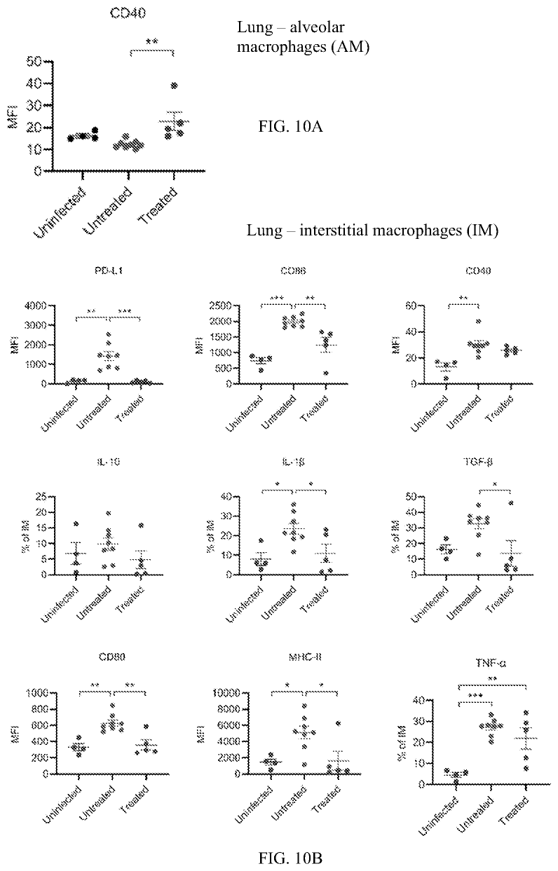

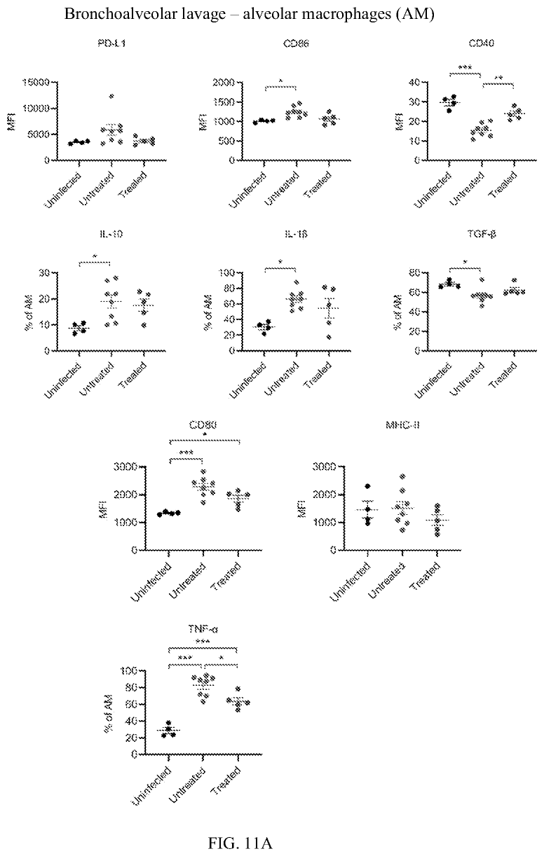

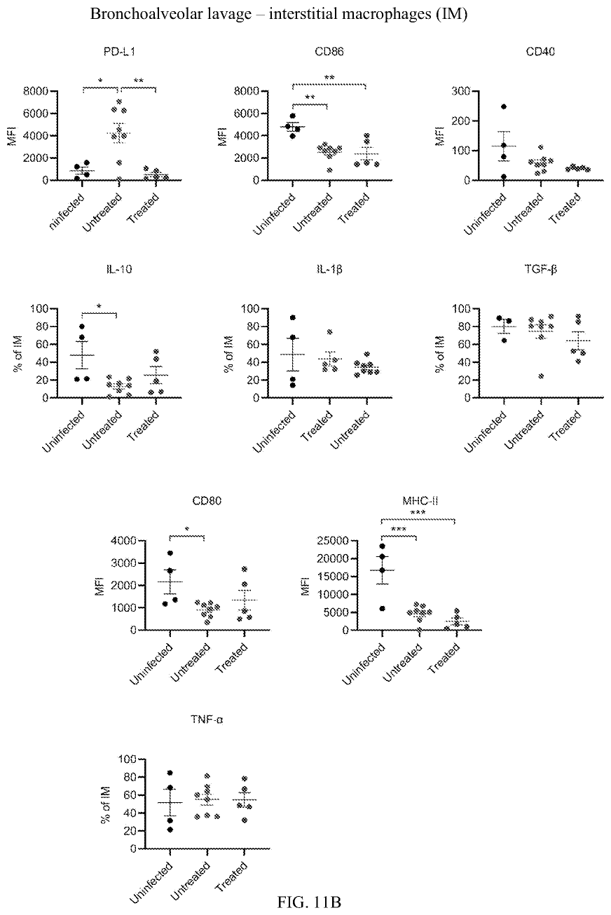

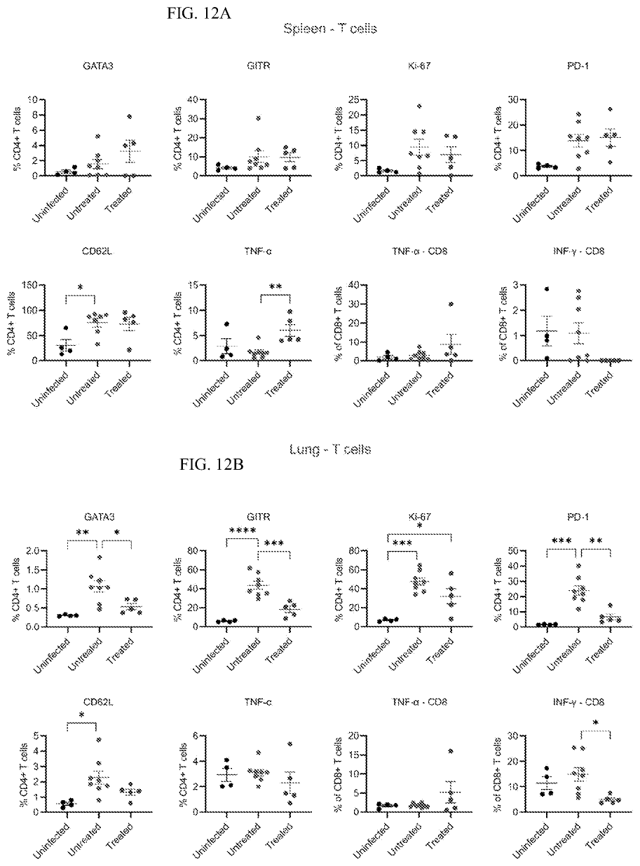

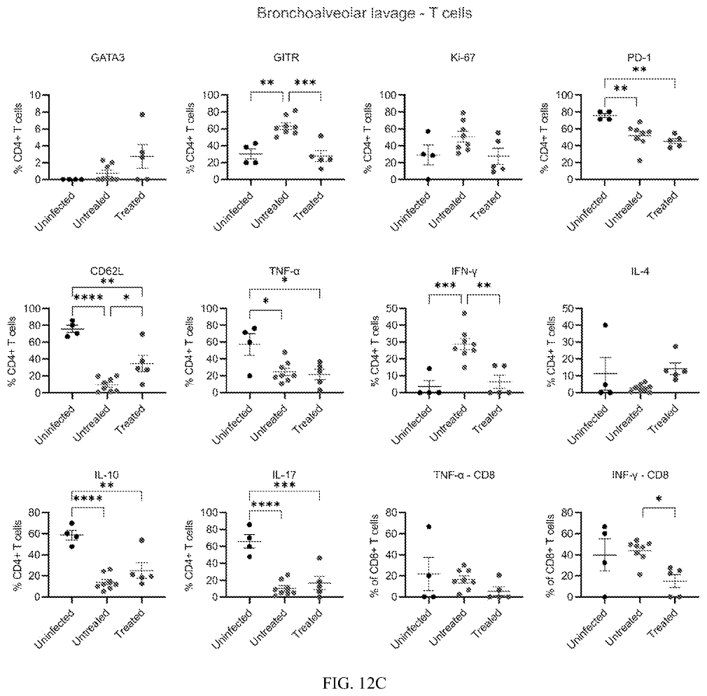

Effects of SFN treatment in the immune response. Given the known immunomodulatory effects of SFN, high-dimensional flow cytometry was employed to evaluate the changes in the immune response of SARS-CoV-2-infected mice treated with SFN and untreated controls, as compared to uninfected mice. Although the immunological landscape was altered as a result of the infection, there were limited differences in overall immune cell composition in the spleen or lungs between treated and untreated mice as visualized using Uniform Manifold Approximation and Projection (UMAP) (FIG. 4A). While changes in the systemic immune responses reflected in the spleen were minimally different (FIGS. 4B-C), there were more pronounced effects locally within the lung (FIGS. 4B, D). Notably, infection induced significant recruitment of myeloid cells, including monocytes and dendritic cells into the lungs of infected mice, however, SFN-treatment significantly reduced this recruitment compared to infected untreated mice (FIG. 4D, P<0.04). Recruitment of blood monocytes into the lung is known to initiate and maintain lung inflammatory responses, including ARDS, and has been demonstrated in SARS-CoV-2 infection (D'Alessio, F. R & Heller, N. M. J Clin Invest 130, 6214-6217 (2020)). SFN treatment significantly decreased the percentage of monocytes and CD11c+ dendritic cells out of total CD45+ immune cells in the lungs (FIG. 5A, P=0.01). Further, metabolically distinct CPT1a+VDAC+ myeloid cells that have been shown to correlate with disease severity in patients with COVID-19 (Thompson, E. A., et al. Metabolic programs define dysfunctional immune responses in severe COVID-19 patients. Cell Rep, 108863 (2021) were significantly decreased in response to SFN treatment (FIG. 5A, P=0.01). Alveolar and interstitial macrophages from the lung of SFN-treated mice displayed lower expression of activation markers such as CD80, CD86, PD-L1, and MHC-II (FIG. 5B, FIGS. 10A-B). Activation also induced significantly lower frequencies of lung alveolar and interstitial macrophages producing cytokines such as IL-10, IL-1β, TNF-α, and TGF-β (FIG. 5C, FIG. 10B, P<0.05). These findings were largely replicated in the bronchoalveolar lavage (FIG. 11). Together, these data highlight the overall reduction of the local myeloid immune responses within the lung microenvironment as a result of SFN treatment. In line with the myeloid compartment, T cell activation was also diminished in response to SFN treatment. Directly ex vivo, CD8+ and CD4+ T cells isolated from the lung of infected untreated mice demonstrated increased expression of activation markers PD1 and MHC-II and the proliferation marker Ki-67, which were significantly decreased in SFN-treated mice (FIG. 5D, P<0.05). This effect on T cell activation was predominantly seen in the lungs and was not found systemically in the spleen. Following stimulation with PMA/ionomycin, CD4+ T cells from the lung, but not the spleen, produced lower levels of IFN-γ and IL-10; however, the frequency of IL-4 and IL-17 were not significantly altered (FIG. 5E, FIG. 12). In summary, the immune-modulatory effects of SFN had a local effect of limiting immune cell activation within the lung, without disturbing or substantially altering systemic immune responses in the spleen.

The ongoing SARS-CoV-2 pandemic has created the immediate need for effective therapeutics that can be rapidly translated to clinical use. Despite the introduction of vaccines, effective antiviral agents are still necessary, particularly considering the potential effects of viral variants (Fontanet, A., et al. The Lancet 397, 952-954 (2021)). New oral antivirals targeting viral enzymes (e.g., molnupiravir and paxlovid) have recently been approved or are in the process of review for emergency use approval by regulatory agencies, with many more currently under development (Jayk Bernal, A., et al. N Engl J Med (2021). December 16; NEJMoa2116044; Owen, D. R, et al. Science 374, 1586-1593 (2021); and Sheahan, T. P., et al. Sci Transl Med 12, eabb5883 (2020). However, this approach can be affected by the emergence of viral variants that change the affinity of the drug to the viral protein (Mascola, J. R, Graham, B. S. & Fauci, A. S. SARS-CoV-2 Viral Variants-Tackling a Moving Target. JAMA 325, 1261-1262 (2021)). An alternative approach is to target host mechanisms required by the virus to infect cells and replicate (Wong, J. P. & Damania, B. Science 371, 884-885 (2021)). Host-directed therapy is advantageous as it allows preexisting drugs to be repurposed, may provide broad-spectrum inhibition against multiple viruses, and is generally thought to be more refractory to viral escape mutations (Bekerman, E. & Einav, S. Science 348, 282-283 (2015); and Wang, R., et al. Cell 184, 106-119.e114 (2021)).

Following exploratory experiments using the in vitro CPE inhibition assay, SFN was identified as a candidate to target the host cellular response, given that it is orally bioavailable, commercially available at low cost, and has limited side-effects (Axelsson, A. S., et al. Sci Transl Med 9, eaah4477 (2017); and Singh, K., et al. Proc Natl Acad Sci USA 111, 15550-15555 (2014)). The results show that SFN has dual antiviral and anti-inflammatory properties against coronaviruses. The results also show that SFN has potent antiviral activity against HCoV-OC43 and multiple strains of SARS-CoV-2, with limited toxicity in cell culture. The similar results observed between the coronaviruses evaluated provide evidence that SFN has broad activity against coronaviruses, a feature that is important as new strains of pathogenic coronaviruses enter the human population. Moreover, synergistic antiviral activity was observed in vitro between SFN and remdesivir against both types of coronaviruses tested; comparable synergism in vivo would be advantageous in clinical scenarios where remdesivir is currently being used. The results demonstrated in vivo efficacy of prophylactic SFN treatment using the K18-hACE2 mouse model of SARS-CoV-2 infection (Winkler, E. S., et al. Nat Immunol 21, 1327-1335 (2020)). Prophylactic SFN-treatment in animals reduced viral replication in the lungs by 1.5 orders of magnitude, similar to that reported for remdesivir in the same mouse model (White, K M., et al. Science 371, 926-931 (2021)). By comparison, BALB/c mice infected with mouse-adapted SARS-CoV-2 had a 1.4 log reduction in viral titers when treated with 300 mg/kg of nirmatrelvir 4 hours after infection (Owen, D. R., et al. Science 374, 1586-1593 (2021)). SFN treatment also modulated the inflammatory response in SARS-CoV-2-infected mice, leading to decreased lung injury.

The pathogenesis of many viral infections is associated with increased production of reactive oxygen species (ROS), which leads to cell death (Lee, C. Oxid Med Cell Longev 2018, U.S. Pat. No. 6,208,067 (2018); Schwarz, KB. Free Radic Biol Med 21, 641-649 (1996); and Siska, P. J., et al. Metabolic imbalance of T cells in COVID-19 is hallmarked by basigin and mitigated by dexamethasone. J Clin Invest 131 (2021)). Conversely, SFN increases antioxidant, anti-inflammatory, and antiviral defenses through multiple mechanisms (Taylor, P. C., et al. Nat Rev Immunol 21, 382-393 (2021); and Jayk Bemal. A., et al. N Engl J Med (2021). December 16; NEJMoa2116044), including the activation of the cap‘n’collar transcription factor NRF2 (Dinkova-Kostova, A. T., et al. Proc Natl Acad Sci USA 99, 11908-11913 (2002)). Under normal conditions, NRF2 remains in an inactive state by association with its inhibitor protein Kelch-like ECH-associated protein 1 (KEAP1)(Hayes, J. D. & Dinkova-Kostova, A. T. Trends Biochem Sci 39, 199-218 (2014)). In response to oxidative stress, KEAP1 is inactivated, and NRF2 is released to induce NRF2-responsive genes that subsequently protect against stress-induced cell death (Soares, Miguel P. & Ribeiro, Ana M. Biochem Soc Trans 43, 663-668 (2015)). SFN has been extensively studied in humans for its anti-cancer properties, has been shown to activate the NRF2 pathway in upper airways (Riedl, M. A., Saxon, A. & Diaz-Sanchez, D. Clin Immunol 130, 244-251 (2009)), and improves the phagocytic ability of alveolar macrophages (Harvey, C. J., et al. Sci Transl Med 3, 78ra32-78ra32 (2011)). The dual antiviral and anti-inflammatory properties of SFN have also been previously described for other viral infections. In vitro antiviral activity has been reported against influenza virus (Kesic, M. J., et al., Free Radic Biol Med 51, 444-453 (2011)), and SFN treatment significantly limited lung viral replication and virus-induced inflammation in respiratory syncytial virus-infected mice (Cho, H.-Y., et al. Am J Respir Crit Care Med 179, 138-150 (2009)).

SFN also inhibits inflammation through NRF-2 independent pathways, such as reducing the proinflammatory nuclear factor kappa B (NF-κB) (Kivela, A. M., et al. Atherosclerosis 213, 122-128 (2010)). NF-κB activation has been described as an important component of the inflammatory response to multiple viral infections, including COVID-19 (Kircheis, R, et al. Front Immunol 11(2020)). There are also other pathways affected by SFN (e.g., STING, STAT3, macrophage migration inhibitory factor) that could play a role in its antiviral response to coronaviruses (Olagnier, D., et al. Nat Commun 9, 1-13 (2018)). While NRF-2 activation and enhanced transcription of its target genes usually requires longer periods of time, potent antiviral activity in cells that had been treated with SFN for 1-2 hours was observed. In NRF2-KD cells infected with SARS-CoV-2, SFN treatment was still able to significantly reduce the viral load. Therefore, the antiviral effect of SFN may be NRF-2 independent while the anti-inflammatory effects are mediated primarily by NRF2.

As a potent NRF2 activator, SFN can modulate the host's immune response while also providing direct, NRF2-independent antiviral effects. Targeting the NRF2 pathway is an approach to develop therapeutics for COVID-19 for multiple reasons (Olagnier, D., et al. Nat Commun 11, 4938 (2020); Herengt, A., et al. Antioxidants 10, 1491 (2021); and Forcados, G. E., et al. Front Cell Infect Microbiol 11(2021)). NRF2 deficiency is known to upregulate the angiotensin-converting enzyme 2 (ACE2), the primary mechanism of cell entry for SARS-CoV-2. The NRF2 activator oltipraz reduces ACE2 levels, suggesting that NRF2 activation might reduce the availability of ACE2 for SARS-CoV-2 entry into the cell (Zhao, S., et al. Endocrinology 159, 836-852 (2018)). Increased NRF2 activity also reportedly inhibits IL-6 and IL-1s gene expression (Kobayashi, E. H., et al. Nat Commun 7, 11624 (2016)), two cytokines known to play important roles in promoting the hyperactive immune response in severely ill COVID-19 patients (Moore, J. B. & June, C. H. Science 368, 473-474 (2020)). Conversely, NRF2 activity is dysregulated in disease states that have been associated with increased severity of COVID-19 (e.g., diabetes)(Rabbani, P. S., et al. Diabetes 68, 141-155 (2019)). Further, NRF2 activity declines in older patients who are more susceptible to severe COVID-19 (Schmidlin, C. J., et al. Free Radic Biol Med 134, 702-707 (2019)). Recent reports suggest that NRF2-dependent genes are suppressed in SARS-CoV-2 infected cells and lung biopsies from COVID-19 patients (Olagnier, D., et al. Nat Commun 11, 4938 (2020)). Similarly, treatment of cells with NRF2 agonists 4-octyl-itaconate and dimethyl fumarate inhibited replication of SARS-CoV-2 in vitro (Olagnier, D., et al. Nat Commun 11, 4938 (2020)).

In contrast to therapeutics that inhibit a single cytokine (e.g., IL-6, IL-10, etc.) (Group, T.W.R.E.Af.C.-T.W. JAMA 326, 499-518 (2021); and Salama, C., et al. N Engl J Med 384, 20-30 (2020)), SFN has important and diverse effects in modulating the lung immune response to SARS-CoV-2 infection. Excessive inflammatory response to SARS-CoV-2 leads to severe disease or death in patients with COVID-19 (Mehta, P., et al. Lancet 395, 1033-1034 (2020)). Therefore, promoting a balanced and robust antiviral response while modulating excessive innate inflammatory responses could represent a favorable scenario that could reduce viral load while also limiting collateral damage to the infected lung. As has been previously reported, SARS-CoV-2 infection leads to an increase in pulmonary dendritic cells and a reduction in CD4+ T cells in K18-hACE2 mice (Winkler, E. S., et al. Nat Immunol 21, 1327-1335 (2020)). Substantial accumulation of immune cells was observed in the lungs of SARS-CoV-2 infected mice, consistent with what has been noted on postmortem analysis of patients with COVID-19 (Winkler, E. S., et al. Nat Immunol 21, 1327-1335 (2020); and Xu, Z., et al. Lancet Respir Med 8, 420-422 (2020)), as well as decreased numbers of T cells in the spleen, consistent with human studies where lymphopenia is correlated with severe COVID-19 (Wang, F., et al. J Infect Dis 221, 1762-1769 (2020)). SFN treatment had significant effects on multiple immune cell populations in the lungs, with a reduction in monocytes, NK cells, and dendritic cells compared to infected untreated controls. These findings are likely the effect of a combination of the overall reduced inflammation and direct effects of SFN on specific cell populations. For example, NK cells exposed to SFN had increased cell lytic function through dendritic cell-mediated IL-12 production (Singh, S. V., et al. Cancer Res 69, 2117-2125 (2009)). The results show decreased recruitment of myeloid cells to the lungs of treated mice and decreased activation profile of local macrophages. The presence of alveolar macrophages with transcriptionally upregulated inflammatory genes and increased secretion of IL-1p have been associated with worse outcomes and increased mortality in patients with ARDS (D'Alessio, F. R. & Heller, N. M. J Clin Invest 130, 6214-6217 (2020); Jacobs, R. F., et al. Am Rev Respir Dis 140, 1686-1692 (2012); Donnelly, S. C., et al. Lancet 341, 643-647 (1993); and Morrell, E. D., et al. Am J Respir Crit Care Med 200, 732-741 (2019)). The results show increased IL-1β in alveolar macrophages with SARS-CoV-2 infection, which was abrogated by SFN treatment (P<0.0001). Mechanistically, the benefits of SFN therapy in the model described herein could be attributed to its modulatory effects on myeloid cells after SARS-CoV-2 infection. SFN treatment led to a reduction in TNF-α in alveolar macrophages and IFN-γ in T cells, both of which are important triggers of cell death and mortality in SARS-CoV-2 infection and cytokine shock syndromes (Karki, R, et al. Cell 184, 149-168.el17 (2021)). Further, SFN was able to reduce but not eliminate T cell activation within the lung. This reduction in T cell activation could be a direct effect in T cells or could operate through downregulation of myeloid costimulatory for T cells such as CD80/CD86. SFN might therefore be able to modulate and dampen immune responses without inhibiting immunity important for viral clearance.

The K18-hACE2 mouse model has been previously used to recapitulate features of COVID-19 in humans (Winkler, E. S., et al. Nat Immunol 21, 1327-1335 (2020)) but differences exist. The expression of the hACE2 transgene, however, is non-physiological, resulting in tissue expression levels that are distinct from endogenously expressed ACE2. Sex differences, which are known to occur with SARS-CoV-2 infection, could not be assessed since male animals were used in these experiments (Dhakal, S., et al. mBio 12, e00974-00921 (2021); and Bunders, M. J. & Altfeld, M. Immunity 53, 487-495 (2020)). Finally, the absorption of SFN after oral administration can be modified by the intestinal microbiome (Yagishita, Y., et al. Molecules 24 (2019), leading to potentially variable drug exposures between animals.

The results described herein demonstrate that pharmacologically relevant micromolar concentrations of SFN inhibited viral replication and virus-induced cell death in vitro. Consumption of SFN-rich broccoli sprouts (single oral daily dose equivalent to 200 μmol of SFN) results in a peak plasma concentration (Cₘₐₓ) of 1.9 μM at 2-3h (Atwell, L. L., et al. Mol Nutr Food Res 59, 424-433 (2015); and Ye, L., et al. Clin Chim Acta 316, 43-53 (2002)), and higher steady state levels could be achieved by administering the same dose in two divided doses (Yagishita, Y., et al. Molecules 24 (2019); Atwell, L. L., et al. Mol Nutr Food Res 59, 424-433 (2015); and Egner, P. A., et al. Cancer Prev Res (Phila) 7, 813-823 (2014)). By comparison, SFN inhibited in vitro SARS-CoV-2 replication in human cells with an IC₅₀ of 2.4 μM. It is important to note that the bioavailability of SFN in humans is dependent on many factors including amount consumed, dietary form and preparation technique, and the individual's gastrointestinal microflora (Yagishita, Y., et al. Molecules 24 (2019); Fahey, J. W. & Kensler, T. W. Front Nutr 8, 648788 (2021); and Shapiro, T., et al. Cancer Epidemiol Biomarkers Prev 7, 1091-1100 (1998)). Studies using SFN-rich broccoli sprouts corresponding to 50 to 400 μmol SFN daily have shown that SFN is well tolerated without clinically significant adverse effects ((Yagishita, Y., et al. Molecules 24 (2019); Singh, K., et al. Proc Natl Acad Sci USA 111, 15550-15555 (2014); Shapiro, T. A., et al. Nutr Cancer 55, 53-62 (2006); and Yagishita, Y., et al. Antioxidants 9, 716 (2020)). Additionally, while SFN is rapidly eliminated from plasma, it reportedly exerts a sustained effect on gene expression (Hu, R., et al. J Pharmacol Exp Ther 310, 263-271 (2004)). A daily dose of SFN-rich broccoli sprouts corresponding to 400 μmol (70 mg) of SFN in humans is not equivalent to the 30 mg/kg of SFN used in the current mouse studies.

In summary, the results show that SFN can inhibit in vitro and in vivo replication of SARS-CoV-2 at pharmacologically and therapeutically achievable concentrations. Further, it can modulate the inflammatory response, thereby decreasing the consequences of infection in mice when administered prior to infection. Given that SFN is orally bioavailable, commercially available, and has limited side effects, the results provide evidence for using SFN for the prevention and treatment of COVID-19 as well as other coronavirus infections.

Materials and Methods. Drugs. L-SFN, 10 mg/mL in ethanol (56 mM), was obtained from Cayman Chemical (Ann Arbor, MI). D,L-SFN was obtained from Millipore Sigma (St. Louis, MO), and a stock solution of 5 mM was prepared in DMSO. Remdesivir was obtained from MedChemExpress or Cayman Chemical, and stock solutions, 5 or 20 mM, respectively, were prepared in DMSO. Drug stock solutions were stored at −25° C.

Cells and viruses. Cells were obtained from the American Type Culture Collection (ATCC, Manassas, VA, USA) and, with the exception of the Vero C1008 cells derived from African green monkeys, are of human origin (Table 2). HCT-8 [HRT-18] (ATCC CCL-244) and Vero C1008 [Vero 76, clone E6, Vero E6] (ATCC CRL-1586) cells were used for growing virus stocks and determining stock titers. Vero C1008 cells, MRC-5 (ATCC CCL-171) cells, and Caco-2 (ATCC HTB-37) cells were used as host cells in antiviral assays. HCT-8 cells were grown in RPMI-1640 medium supplemented with 10% fetal bovine serum (FBS) (MilliporeSigma. St. Louis, MO, USA), L-glutamine, penicillin-streptomycin, and sodium pyruvate. Vero C1008 and MRC-5 cells were grown in Eagles Minimum Essential Medium (EMEM) with 10% FBS, L-glutamine, and penicillin-streptomycin at 37° C. with 5% CO₂. Caco-2 cells were grown in EMEM supplemented with 10% FBS, lx sodium pyruvate, L-glutamine, and penicillin-streptomycin at 37° C. with 5% CO₂.

TABLE 2
List of evaluated cell lines.

| | Source | | | | |
|---|---|---|---|---|---|
| | (Catalog | | | Morphol- | |
| Cell line* | number) | Organism | Tissue | ogy | Disease |
| HCT-8 | ATCC^{#} | Human | Colon | Epithelial | Adenocar- |
| [HRT-18] | (CCL-244) | | | | cinoma |
| Caco-2 | ATCC | Human | Colon | Epithelial | Adenocar- |
| | (HTB-37) | | | | cinoma |
| MRC-5 | ATCC | Human | Lung | Fibroblast | Normal |
| | (CCL-171) | | | | |
| Vero C1008 | ATCC | African | Kidney | Epithelial | Normal |
| [Vero 76, | (CRL-1586) | green | | | |
| clone E6, | | monkey | | | |
| Vero E6] | | | | | |

*None of the cell lines listed are registered as a misidentified cell line according to the International Cell Line Authentication Committee (ICLAC) Register of Misidentified Cell Lines, version 11. iclac.org/databases/cross-contaminations/
^{#}ATCC, American Type Culture Collection.

Human coronavirus OC43 (HCoV-OC43) was purchased from ATCC (Betacoronavirus 1, ATCC VR-1558). SARS-CoV-2/Wuhan-1/2020 virus (U.S. Centers for Disease Control and Prevention) was provided by Dr. Andrew Pekosz (Johns Hopkins). 2019-nCoV/USA-WA1/2020 was obtained through BEI Resources, National Institute of Allergy and Infectious Diseases (NIAID), National Institutes of Health (NIH). Two 614G+ clinical strains of SARS-CoV-2, SARS-CoV-2/USA/DCHP-7/2020 (DC), and SARS-CoV-2/USA/MDHP-20/2020 (MD) were isolated from patients at. The Johns Hopkins Hospitals (Gniazdowski, V., et al. Clin Infect Dis 73, e860-e869 (2020)). The virus stocks were stored at −80° C., and titers were determined by tissue culture infectious dose 50 (TCID₅₀) assay.

CRISPRCas9 knockdown and Western blot. For the generation of the NRF2 KD cells, Caco-2 cells were co-transfected with NRF2-specific CRISPR/Cas9 (sc-400017) and HDR plasmids (sc-400017-HDR) using the UltraCruz® Transfection Reagent (sc-395739), as recommended by the manufacturer (Santa Cruz Biotechnology). Control cells were transfected with the HDR plasmid. Cells were selected for 2 weeks with media containing puromycin (3 μg/ml). NRF2 knockdown efficiency was assayed by Western blot (Merino, V. F., et al. Combined Treatment with Epigenetic, Differentiating, and Chemotherapeutic Agents Cooperatively Targets Tumor-Initiating Cells in Triple-Negative Breast Cancer. Cancer Res 76, 2013-2024 (2016) using an NRF2 antibody from Abcam (1:1000 dilution, ab62352).

Cytopathic effect (CPE) inhibition assay. An assay protocol was used that interrogates both antiviral and anti-host cell activities to evaluate compounds (Revu, O., et al. ChemistrySelect 1, 5895-5899 (2016)). This assay is predicated upon the virus's ability to cause a CPE, measured in TCID₅₀. Host cells, 7.5-10×10³ in virus growth medium (VGM; Dulbecco's Modified Eagle Medium without phenol red supplemented with 3% FBS), were plated in clear 96-well half-area tissue cultures plates or white, clear-bottom 96 well plates, 24 hours prior to the assay. On the day of the assay, working solutions of drugs (0.1-1 mM) were made by dilution of drug stocks in VGM. For one-drug analysis assays, 50 μL of the drug working solution was added to each well in the first column of cells, and then drugs were serially diluted across the plate by dilutions of 0.5 log₁₀. The default drug test range was 320-0.032 μM. Drug-exposed cells were incubated for 1-24 hours at 35° C. (HCoV-OC43) or 37° C. (SARS-CoV-2), after which time 32 (HCoV-OC43; SARS-CoV-2) or 50 (SARS-CoV-2) TCID₅₀ of virus suspended in VGM or VGM alone was added to cells. Test plates had virus control wells (virus+/drug−), drug control wells (virus−/drug+), and cell control wells (virus−/drug−). After 3-4 days incubation at 35° C. or 37° C./5% CO₂, the cell viability was assessed using Celltiter 96®AQᵤₑₒᵤₛ One Solution (Promega Corp, WI, USA) or CellTiter-Glo® One Solution Assay system (Promega) following manufacturer protocols. Color reactions were read at 490-650 nm absorbance in a Filtermax F5 microplate reader (Molecular Devices, CA, USA) using SoftMax Pro 6.5 software. Luminescence readouts were obtained in a FLUOstar Omega plate reader (BMG Labtech, Ortenberg, Germany). For two-drug combination assays, one test drug was serially diluted across the plate (left to right) as described herein; the second drug was serially diluted down the plate (top to bottom). The starting concentration of the first drug was adjusted to allow for dilution with the second drug. For interrogation of a drug's ability to affect an established viral infection, host cells were infected with the virus and allowed to incubate for 24 hours. After this time, drug dilutions that resulted in the concentration ranges mentioned above, or VGM, were added to appropriate wells. Cell viability reagent was added at 3-4 days post-infection. For examination of a drug's ability to prevent an infection by pretreatment of the host cells, the test drug was serially diluted across the plate as described herein. After 24 hours incubation, the drug was removed by aspiration, the cells were rinsed once with warm Hanks Balanced Salt Solution, and then 32 TCID₅₀ of virus in drug-free VGM was added to appropriate wells. Cell viability reagent was added at 4 days post-infection.

In vitro data analysis. Calcusyn software (Biosoft, Cambridge, UK) was used to calculate the median inhibitory concentration (IC₅₀), median cytotoxic dose (TD₅₀), and to generate median effect plots and dose-response curves. The therapeutic index (TI), a measure of antiviral selectivity, was calculated by the formula TI=TD₅₀/IC₅₀. Combination Indices (CI) for two-drug combination assays were calculated by Calcusyn software. Isobolograms to depict synergistic, additive, and antagonistic combinations were generated by the software.

Viral RNA determination. Zymo Quick-RNA Viral 96 Kit (Zymo Research) was used to isolate RNA from cell supematants according to the manufacturer's protocol. cDNA synthesis was performed using qScript cDNA Supermix containing random hexamers and oligo-dT primers following the manufacture's protocol (Quanta Biosciences). Real-time quantitative reverse transcription

PCR (RT-qPCR) was performed in technical triplicate for each sample using TaqMan Fast Advanced Master Mix (Applied Biosystems) on a StepOne Plus Real Time PCR machine (Applied Biosystems). Primers and probes are listed below. The cycling parameters were as follows: (i) 2 min at 50° C.; (ii) 2 min at 95° C.; and (iii) 45 cycles at 95° C. for 3 s and 55° C. for 30s. Molecular standard curves were generated using serial dilutions of a plasmid containing the complete SARS-CoV-2 N gene (Integrated DNA Technologies, Catalog #10006625). SARS-CoV-2 RNA was detected using premixed forward (5′-TTACAAACATTGGCCGCAAA-3′; SEQ ID NO: 1) and reverse (5′-GCGCGACATTCCGAAGAA-3′; SEQ ID NO: 2) primers and probe (5′-FAM-ACAATTGCCCCCAGCGCTTCAG-BHQ1-3′; SEQ ID NO: 3) designed by the U.S. CDC as part of the COVID-19 CDC Research Use Only kit (Integrated DNA Technologies, Catalog #: 10006713) to amplify a region of the SARS-CoV-2 nucleocapsid (N) gene. For lung tissues, the sample was homogenized in 1 mL of TRIzol (Invitrogen), and the RNA was isolated using a combined protocol of TRIzol phenol chloroform and the RNeasy Mini Kit (QIAGEN) according to the manufacturer's protocol, and RT-qPCR was performed as described herein. For lung tissue lysates, viral copies per lung sample were normalized to the relative expression of the mouse RNA Polymerase II gene (Pol2Ra) using the TaqMan gene expression assay (Catalog #: Mm00839502_ml; ThermoFisher) (Radonić, A., et al. Biochem Biophys Res Commun 313, 856-862 (2004)).

Animal experiments. Heterozygous K18-hACE2 C57BL/6J mice (strain: 2B6.Cg-Tg(K18-ACE2)2Prlmn/J) were obtained from The Jackson Laboratory and propagated at Johns Hopkins University School of Medicine. Animals were separately housed in groups and fed standard chow diets. Male mice, 6-8 weeks old, were used for this study. A subgroup of animals received 30 mg/kg daily of SFN diluted in 2% ethanol in water via oral gavage. Treatment started one day prior to viral infection. Infected untreated and uninfected controls also received daily oral gavage with 2% ethanol in water. After induction of anesthesia with ketamine hydrochloride and xylazine, the animals received 8.4×10⁵ TCID₅₀ of SARS-CoV-2/USA/WI1/2020 intranasally. Uninfected animals received intranasally the same volume of vehicle. Weights were monitored daily, the animals were sacrificed 6 days post-infection by isoflurane overdose, and the tissues were harvested. Tissues were perfused with PBS after serum collection via cardiac puncture and before tissue harvest Bronchoalveolar lavage (BAL) was obtained by cannulating the trachea with a 20-gauge catheter. The right lung was lavaged twice (each aliquot 1 ml; calcium-free PBS); total returns averaged 1-1.5 ml/mouse. BAL was centrifuged at 600 g for 8 minutes at 4° C. The cell-free supernatants were stored at −80° C. for total protein quantification using the BCA protein assay (Sigma).

Flow cytometry. Lungs were minced and incubated at 37° C. in an enzyme cocktail of RPMI containing 2.4 mg/mi collagenase I and 20 μg/ml DNase (Invitrogen), then mashed through a 70-μm nylon cell strainer (BD Falcon). The flow cytometry antibodies used for phenotypic and metabolic analysis can be found in Table 3. For analysis immediately ex vivo, cells were washed once in PBS and immediately stained for viability with Biolegend Live/Dead Zombie NIR Fixable Viability Dye and Fc Block for 10 min at room temperature. Cell surface staining was performed in 100 μL of 20% BD Horizon™ Brilliant Stain Buffer+ PBS with surface stain antibody cocktail for 20 min at room temperature. Cells were fixed and permeabilized with eBioscience™ FoxP3/Transcription Factor Staining kit 1× Fixation/Permeabilization reagent overnight at 4° C. Cells were washed with 1× Permeabilization/Wash buffer. Intracellular staining (ICS) was performed in 100 μL 1× Permeabilization/Wash buffer with ICS antibody cocktail for 45 min at room temperature. Cells were washed once with Permeabilization/Wash buffer then resuspended in Permeabilization/Wash buffer for acquisition by flow. To improve the quality of the T-cell flow cytometry functional staining, the cells were stimulated with phorbol 12-myristate 13-acetate (PMA, 50 ng/mL) and inomycin (1 μg/mL) for 1 h, following for a 3 h incubation with protein transport inhibitors (GolgiPlug and GolgiStop, BD). For the myeloid flow cytometry functional staining, the cells were incubated with protein transport inhibitors for 4 h. Samples were run on a 3 laser Cytek Aurora spectral flow cytometer or a FACSAria II spectral flow cytometer (BD). FCS files were analyzed using Flowjo v10.6.2 software (BD). Manual gating strategies for the panels can be found in FIG. 8. High-dimensional unbiased analysis of cell phenotypes was performed using Flowjo plugins DownSample v3 and UMAP.

TABLE 3
List of antibodies used for flow cytometry.

| | | | | |
|---|---|---|---|---|
| Marker | Fluorophore | Vendor | Catalog # | Clone |

Ex vivo panel

| | | | | |
|---|---|---|---|---|
| NK1.1 | PE CF594 | BD Biosciences | 562864 | PK136 |
| CD19 | PE Cy5 | BioLegend | 115510 | 6D5 |
| CD62L | PECy7 | BD Biosciences | 560516 | MEL-14 |
| CD11b | AF700 | BioLegend | 101222 | M1/70 |
| CD4 | APC Cy7 | BD Biosciences | 565650 | RM4-5 |
| Ly6G | efluor 450 | ThermoFisher | 48-5931- | RB6-8C5 |
| | | | 82 | |
| CD103 | BV480 | BD Biosciences | 566201 | M290 |
| CD44 | BV510 | BioLegend | 103044 | IM7 |
| Ly6c | BV570 | BioLegend | 128030 | HK1.4 |
| PD1 | BV605 | BioLegend | 135220 | 29F.1A12 |
| TCRb | BV650 | BioLegend | 109251 | H57-597 |
| MCHII | BV711 | BioLegend | 107643 | M5/114.15.2 |
| CD11c | BV750 | BioLegend | 117357 | N418 |
| F4/80 | BV785 | BioLegend | 123141 | BM8 |
| CD69 | BUV737 | BD Biosciences | 612793 | H1.2F |
| CD8 | BUV805 | BD Biosciences | 612898 | 53-6.7 |
| CD45 | Super | Fischer | 62045182 | 30-F11 |
| | Bright 436 | Scientific | | |
| CPT1a | AF488 | Abcam | ab171449 | 8F6AE9 |
| VDAC1 | AF532 | Abcam | ab14734 | 20B12AF2 |
| H3K27Me3 | PE | CST | 40724 | C36B11 |
| FOXP3 | PerCpCy5.5 | ThermoFisher | 45-5773- | FJK-16x |
| | | | 82 | |
| Ki67 | PerCp- | ThermoFisher | 46-5698- | SolA15 |
| | eFluor710 | | 82 | |
| GLUT1 | AF647 | Abcam | ab195020 | EPR3915 |
| Hexokinase | Dylight680 | Abcam | ab228819 | EPR20839 |
| II | | | | |
| Tomm20 | AF405 | Abcam | ab210047 | EPR15581- |
| | | | | 54 |

Macrophage panel

| | | | | |
|---|---|---|---|---|
| CD80 | FITC | BioLegend | 104716 | 16-10A1 |
| CD11B | PE-CF594 | ThermoFisher | RM2817 | M1/70.15 |
| CD64 | PE-Cy7 | BioLegend | 139314 | X54-5/7.1 |
| CCR2 | APC | BioLegend | 150604 | SA203G11 |
| MHC-II | APC-Cy7 | BioLegend | 107628 | M5/114.15.2 |
| CD11C | APC-R700 | BD Biosciences | 565872 | N418 |
| LY6C | BV605 | BioLegend | 128036 | HK1.4 |
| CD86 | BV650 | BD Biosciences | 564200 | GL1 |
| CD40 | BV786 | BD Biosciences | 740891 | 3/23 |
| CD45R-B | BV750 | BioLegend | 103261 | RA3-6B2 |
| B7-H1 | BV711 | BD Biosciences | 563369 | MIH5 |
| CD24 | BUV737 | BD Biosciences | 565308 | M1/69 |
| SigF | BUV395 | BD Biosciences | 740280 | E50-2440 |
| IL-10 | PE | BioLegend | 505008 | JES5-16E3 |
| IL-1B | PerCP | ThermoFisher | 46-7114- | NJTEN3 |
| | | | 82 | |
| TGF-B | BV421 | BD Biosciences | 565638 | TW7-16B4 |
| TNF-a | BV510 | BD Biosciences | 563386 | MP6-XT22 |

T cell panel

| | | | | |
|---|---|---|---|---|
| CD62L | APC-R700 | BD biosciences | 565159 | MEL-14 |
| TCR | APC-750 | BioLegend | 109246 | H57-597 |
| PD-1 | BV605 | BioLegend | 135220 | 29F.1A12 |
| CD25 | BV650 | BD Biosciences | 564021 | PC61 |
| CD357 | BV711 | BD Biosciences | 563390 | DTA-1 |
| (GITR) | | | | |
| CD45R-B | BV750 | BioLegend | 103261 | RA3-6B2 |
| CD3 | BV785 | BioLegend | 100355 | 145-2C11 |
| CD4 | BUV395 | BD Biosciences | 563790 | GK1.5 |
| CD8 | BUV737 | BD Biosciences | 612759 | 53-6.7 |
| IL-17A | AF 488 | BioLegend | 506910 | TC11- |
| | | | | 18H10.1 |
| IL-10 | PE | BioLegend | 505008 | JES5-16E3 |
| Ki-67 | PerCPe710 | ThermoFisher | 46-5698- | SolA15 |
| | | | 82 | |
| IFN-y | PE-Cy7 | BioLegend | 505826 | XMG1.2 |
| GATA3 | PE-CF594 | BD Biosciences | 563510 | L50-823 |
| FOXP3 | APC | ThermoFisher | 17-5773- | FJK-16s |
| | | | 82 | |

Histology and immunohistochemistry. After euthanasia, tissues were fixed on 10% neutral-buffered formalin. Tissues were embedded in paraffin and sections were stained with hematoxylin and eosin. Digital light microscopy scans of the lung were examined implementing a semi-quantitative, 5-point grading scheme (0—within normal limits, 1—mild, 2—moderate, 3—marked, 4—severe) (White, K. M., et al. Science 371, 926-931 (2021)). The scoring system considered four different histopathological parameters: 1) perivascular inflammation, 2) bronchial or bronchiolar epithelial degeneration or necrosis, 3) bronchial or bronchiolar inflammation, and 4) alveolar inflammation. These changes were absent (grade 0) in lungs from uninfected mice. Individual total pathology scores varied from 1/16 to 2/16 for the SFN-treated group and from 4/16 to 9/16 for the infected untreated controls. Immunostaining was performed at the Oncology Tissue Services Core of Johns Hopkins University School of Medicine. Immunolabeling for the SARS-CoV-2 spike protein was performed on formalin-fixed, paraffin-embedded sections on a Ventana Discovery Ultra autostainer (Roche Diagnostics). Briefly, following dewaxing and rehydration on board, epitope retrieval was performed using Ventana Ultra CC1 buffer (catalog #6414575001, Roche Diagnostics) at 96° C. for 64 minutes. Primary antibody, anti-SARS-CoV-2 spike protein (1:200 dilution; catalog #GTX135356, lot #43957, Genetex) was applied at 36° C. for 60 minutes. Primary antibodies were detected using an anti-rabbit HQ detection system (catalog #7017936001 and 7017812001, Roche Diagnostics) followed by Chromomap DAB IHC detection kit (catalog #5266645001, Roche Diagnostics), counterstaining with Mayer's hematoxylin, dehydration, and mounting. The primary antibody for hACE2 immunolabeling was recombinant Anti-ACE2 antibody [Catalog #EPR4435(2), abl08252; Abcam]. Automated analysis of the SARS-CoV-2 spike protein immunostaining was performed with Halo v3.2.1851.328 (Indica Labs). The area corresponding to the anti-SARS-CoV-2 spike protein was divided by the sum of the areas corresponding to cellular structures stained with Mayer's hematoxylin and anti-SARS-CoV-2 spike protein. The ratio is represented as a percentage.

Statistics and reproducibility. Data were analyzed using Prism 9.2.0 (GraphPad). Specifics of statistical comparisons are detailed in individual figure legends. Statistical significance was assigned when P values were <0.05.

What is claimed is:

1. A method of treating a subject having a coronavirus infection, the method comprising administering to the subject one or more therapeutically effective doses of sulforaphane.

2. The method of claim 1, wherein sulforaphane inhibits replication of the coronavirus.

3. A method of treating or preventing COVID-19 in a subject, the method comprising administering to the subject one or more therapeutically effective doses of sulforaphane.

4. A method of preventing or inhibiting a coronavirus infection in a subject, the method comprising administering to the subject one or more therapeutically effective doses of sulforaphane.

5. A method of inhibiting replication of a coronavirus in a subject having a coronavirus infection, the method comprising administering to the subject one or more therapeutically effective doses of sulforaphane.

6. The method of any of the preceding claims, further comprising administering to the subject one or more therapeutically effective doses of remdesivir.

7. A method of inhibiting replication of a coronavirus in a cell, the method comprising contacting the cell infected with the coronavirus with one or more therapeutically effective doses of sulforaphane.

8. The method of any of the preceding claims, wherein the sulforaphane is administered in a composition comprising at least one pharmaceutically acceptable carrier, diluent or excipient.

9. The method of any of claims 1-6 or 8, wherein the administration is systemic, buccal, parenteral, enteral, intramuscular, subdermal, sublingual, peroral, oral, or a combination thereof.

10. The method of any of the preceding claims, wherein the subject is infected or has previously been infected with the coronavirus.

11. The method of any of the preceding claims, wherein the coronavirus is SARS-CoV, MERS-CoV, SARS-CoV-2, HCoV 229E, HCoV NL63, HCoV OC43, HCoV HKU1,

12. The method of any of the preceding claims, wherein the sulforaphane total dose per day is independently selected upon each occurrence from about 25 μmol to about 200 μmol.

13. The method of claim 6, wherein the therapeutically effective dose of sulforaphane and remdesivir are in a ratio of 1:01 to 1:10.

14. The method of claim 6, wherein the therapeutically effective doses of sulforaphane and remdesivir are administered in synergistic combination.

15. A method of inhibiting, treating or preventing a coronavirus infection in a subject, the method comprising administering to the subject having said infection a plurality of therapeutically effective doses of sulforaphane.

16. The method of claim 15, wherein the plurality of therapeutically effective doses of sulforaphane is one or more doses administered per day for two or more days per week.

17. The method of claim 16, wherein dosing is continued for one or more weeks per month.

18. The method of claim 17, wherein dosing is continued for one or more months per year.

19. The method of claim 15, wherein the coronavirus is pathogenic to humans.

20. The method of claim 19, wherein the coronavirus is SARS-CoV, MERS-CoV, SARS-CoV-2, HCoV 229E, HCoV NL63, HCoV OC43, or HCoV HKU1.

21. The method of claim 15, wherein the administration is systemic, buccal, enteral, parenteral, intramuscular, subdermal, sublingual, peroral, oral, or a combination thereof.

22. The method of claim 15, further comprising administering a therapeutic effective amount of remdesivir to the subject.

23. The method of claim 15, wherein the sulforaphane is administered to the subject immediately after infection or any time within one day to 5 days after infection or at the earliest time after diagnosis of infection with the coronavirus.

24. The method of claim 15, wherein the sulforaphane is administered to the subject as a primary antiviral therapy, adjunct antiviral therapy, or a co-antiviral therapy, or wherein the administration comprises separate administration or coadministration of sulforaphane with at least one other antiviral composition or with at least one other composition for treating one or more symptoms associated with said coronavirus infection.

25. A kit for use in treating a subject suffering from a coronavirus infection, said kit comprising: (a) sulforaphane; and (b) remdesivir.

26. A kit for use in treating a subject suffering from a coronavirus infection, said kit comprising: (a) sulforaphane; and (b) molnupiravir, 4′-fluorouridine, favipiravir, remdesivir, nirmatrelvir, ritonavir, a combination of nirmatrevlir and ritonavir, GC-376, cepharanthine, cefoperazone, dihydroergotamine, cefpiramide, ergoloid, ergotamine, netupitant, Dpnh (NADH), lifitegrast, nilotinib, tubocurarin, lumacraftor, emend, irinotecan, enjuvia, zelboraf, cromolyn, diosmin, Risperdal, differin, plitidepsin, convalescent plasma, actemra, recombinant soluble ACE2, camostate mesylate and analogs thereof, or fluvoxamine.

27. A kit for use in preventing or inhibiting a coronavirus infection in a subject, said kit comprising: (a) sulforaphane; and (b) remdesivir.

28. A kit for use in preventing or inhibiting a coronavirus infection in a subject, said kit comprising: (a) sulforaphane; and (b) molnupiravir, 4′-fluorouridine, favipiravir, remdesivir, nirmatrelvir, ritonavir, a combination of nirmatrevlir and ritonavir, GC-376, cepharanthine, cefoperazone, dihydroergotamine, cefpiramide, ergoloid, ergotamine, netupitant, Dpnh (NADH), lifitegrast, nilotinib, tubocurarin, lumacraftor, emend, irinotecan, enjuvia, zelboraf, cromolyn, diosmin, Risperdal, differin, plitidepsin, convalescent plasma, actemra, recombinant soluble ACE2, camostate mesylate and analogs thereof, or fluvoxamine.

29. A kit for use in inhibiting replication of a coronavirus infection in a subject, said kit comprising: (a) sulforaphane; and (b) remdesivir.

30. A kit for use in inhibiting replication of a coronavirus infection in a subject, said kit comprising: (a) sulforaphane; and (b) molnupiravir, 4′-fluorouridine, favipiravir, remdesivir, nirmatrelvir, ritonavir, a combination of nirmatrevlir and ritonavir, GC-376, cepharanthine, cefoperazone, dihydroergotamine, cefpiramide, ergoloid, ergotamine, netupitant, Dpnh (NADH), lifitegrast, nilotinib, tubocurarin, lumacraftor, emend, irinotecan, enjuvia, zelboraf, cromolyn, diosmin, Risperdal, differin, plitidepsin, convalescent plasma, actemra, recombinant soluble ACE2, camostate mesylate and analogs thereof, or fluvoxamine.

31. The kit of claims 25-30, further comprising at least one pharmaceutically acceptable carrier, diluent or excipient.

32. The kit of claim 25-30, further comprising instructions for using sulforaphane in treating a coronavirus infection.

33. The method of any of the preceding claims, further comprising administering to the subject one or more therapeutically effective doses of a polymerase inhibitor.

34. The method of claim 33, wherein the polymerase inhibitor is molnupiravir, 4′-fluorouridine, favipiravir, or remdesivir.

35. The method of claim 33 or 34, wherein the therapeutically effective doses of sulforaphane and the polymerase inhibitor are administered in an additive or synergistic combination.

36. The method of any of the preceding claims, further comprising administering to the subject one or more therapeutically effective doses of a protease inhibitor.

37. The method of claim 36, wherein the protease inhibitor is nirmatrelvir, ritonavir, a combination of nirmatrevlir and ritonavir, or GC-376.

38. The method of claim 36 or 37, wherein the therapeutically effective doses of sulforaphane and the protease inhibitor are administered in an additive or synergistic combination.

39. The method of any of the preceding claims, further comprising administering to the subject one or more therapeutically effective doses of a helicase inhibitor.

40. The method of claim 39, wherein the helicase inhibitor is cepharanthine, cefoperazone, dihydroergotamine, cefpiramide, ergoloid, ergotamine, netupitant, Dpnh (NADH), lifitegrast, nilotinib, tubocurarin, lumacraftor, emend, irinotecan, enjuvia, zelboraf, cromolyn, diosmin, Risperdal, or differin.

41. The method of claim 39 or 40, wherein the therapeutically effective doses of sulforaphane and the helicase inhibitor are administered in an additive or synergistic combination.

42. The method of any of the preceding claims, further comprising administering to the subject one or more therapeutically effective doses of an inhibitor of host proteins supporting viral replication.

43. The method of claim 42, wherein the inhibitor of host proteins supporting viral replication is plitidepsin.

44. The method of claim 42 or 41, wherein the therapeutically effective doses of sulforaphane and the inhibitor of host proteins supporting viral replication are administered in an additive or synergistic combination.

45. The method of any of the preceding claims, further comprising administering to the subject one or more therapeutically effective doses of a non-vaccine biologic.

46. The method of claim 45, wherein the non-vaccine biologic is convalescent plasma, actemra, a monoclonal antibody specific for a viral protein.

47. The method of claim 45 or 46, wherein the therapeutically effective doses of sulforaphane and the non-vaccine biologic are administered in an additive or synergistic combination.

48. The method of any of the preceding claims, further comprising administering to the subject one or more therapeutically effective doses of an inhibitor of viral attachment and entry.

49. The method of claim 48, wherein the inhibitor of viral attachment and entry is human recombinant soluble angiotensin converting enzyme—2 (ACE2) or camostate mesylate and analogs thereof.

50. The method of claim 48 or 49, wherein the therapeutically effective doses of sulforaphane and the inhibitor of viral attachment and entry are administered in an additive or synergistic combination.

51. The method of any of the preceding claims, further comprising administering to the subject one or more therapeutically effective doses of a selective serotonin reuptake inhibitor.

52. The method of claim 51, wherein the selective serotonin reuptake inhibitor is fluvoxamine.

53. The method of claim 51 or 52, wherein the therapeutically effective doses of sulforaphane and the selective serotonin reuptake inhibitor are administered in an additive or synergistic combination.


SEQUENCE LISTING (3 sequences)

SEQ ID NO: 1 - DNA, 20 bp, Artificial Sequence
  Synthetic construct
ttacaaacattggccgcaaa

SEQ ID NO: 2 - DNA, 17 bp, Artificial Sequence
  Synethetic construct
cgcgacattccgaagaa

SEQ ID NO: 3 - DNA, 23 bp, Artificial Sequence
  Synethetic construct
  misc_feature (1)..(1): FAM attached
  misc_feature (23)..(23): BHQ1 attached
acaatttgcccccagcgcttcag